(19) United States
(12) Patent Application Publication
(10) Pub. No.: US 2008/0124272 A1
(43) Pub. Date: May 29, 2008

(54) Influenza antibodies, compositions, and related methods

(76) Inventors: Vidadi Yusibov, Havertown, PA (US); Gene Palmer, New Castle, DE (US); Vadim Mett, Newark, DE (US)

Correspondence Address: CHOATE, HALL & STEWART LLP, TWO INTERNATIONAL PLACE, BOSTON, MA, 02110, US

(21) Appl. No.: 11/707,257

(22) Filed: Feb. 13, 2007

Related U.S. Application Data
(60) Provisional application No. 60/844,770, filed on Sep. 15, 2006.

(51) Int. Cl. A61K 9/127; A61K 39/395; A61K 49/00; C07K 16/18; C12N 5/16; C07K 16/46; C07K 16/08; A61K 51/12

(52) U.S. Cl. 424/1.49; 424/141.1; 424/450; 424/9.1; 435/326; 435/5; 530/387.3; 530/388.1; 530/388.15

(57) ABSTRACT

The present invention relates to the intersection of the fields of immunology and protein engineering, and particularly to antigens and vaccines useful in prevention of infection by influenza virus. Provided are recombinant protein antigens, compositions, and methods for the production and use of such antigens and vaccine compositions.

### RELATED APPLICATIONS

The present application is related to and claims priority under 35 USC 119(e) to U.S. Ser. No. 60/844,770, filed Sep. 15, 2006 (the '770 application); the entire contents of the '770 application are incorporated herein by reference.

### BACKGROUND OF THE INVENTION

Influenza has a long history characterized by waves of pandemics, epidemics, resurgences and outbreaks. Influenza is a highly contagious disease that could be equally devastating both in developing and developed countries. The influenza virus presents one of the major threats to the human population. In spite of annual vaccination efforts, influenza infections result in substantial morbidity and mortality. Although flu epidemics occur nearly every year, fortunately pandemics do not occur very often. However, recent flu strains have emerged such that we are again faced with the potential of an influenza pandemic. Avian influenza virus of the type H5N1, currently causing an epidemic in poultry in Asia as well as regions of Eastern Europe, has persistently spread throughout the globe. The rapid spread of infection, as well as cross species transmission from birds to human subjects, increases the potential for outbreaks in human populations and the risk of a pandemic. The virus is highly pathogenic, resulting in a mortality rate of over fifty percent in birds as well as the few human cases which have been identified. If the virus were to achieve human to human transmission, it would have the potential to result in rapid, widespread illness and mortality.

The major defense against influenza is vaccination. Influenza viruses are segmented, negative-strand RNA viruses belonging to the family Orthomyxoviridae. The viral antigens are highly effective immunogens, capable of eliciting both systemic and mucosal antibody responses. Influenza virus hemagglutinin glycoprotein (HA) is generally considered the most important viral antigen with regard to the stimulation of neutralizing antibodies and vaccine design. The presence of viral neuraminidase (NA) has been shown to be important for generating multi-arm protective immune responses against the virus. Antivirals which inhibit neuraminidase activity have been developed and may be an additional antiviral treatment upon infection. A third component considered useful in the development of influenza antivirals and vaccines is the ion channel protein M2.

Subtypes of the influenza virus are designated by different HA and NA resulting from antigenic shift. Furthermore, new strains of the same subtype result from antigenic drift, or mutations in the HA or NA molecules which generate new and different epitopes. Although 15 antigenic subtypes of HA have been documented, only three of these subtypes H1, H2, and H3, have circulated extensively in humans. Vaccination has become paramount in the quest for improved quality of life in both industrialized and underdeveloped nations. The majority of available vaccines still follow the basic principles of mimicking aspects of infection in order to induce an immune response that could protect against the relevant infection. However, generation of attenuated viruses of various subtypes and combinations can be time consuming and expensive. Along with emerging new technologies, in-depth understanding of a pathogen's molecular biology, pathogenesis, and interactions with an individual's immune system has resulted in new approaches to vaccine development and vaccine delivery. Thus, while technological advances have improved the ability to produce improved influenza antigens vaccine compositions, there remains a need to provide additional sources of protection against to address emerging subtypes and strains of influenza.

### SUMMARY OF THE INVENTION

The present invention provides antibodies against influenza neuraminidase antigens and antibody components produced in plants. The present invention provides antibodies which inhibit the activity of neuraminidase. The invention further provides antibody compositions reactive against influenza neuraminidase antigen. In some embodiments, provided compositions include one or more plant components. Still further provided are methods for production and use of the antibodies and compositions of the invention.

### BRIEF DESCRIPTION OF THE DRAWING

FIG. 1. Map of the pET32 plasmid. The top left indicates the region between the T7 promoter and the T7 terminator lacking in modified plasmid used for cloning target antigen.

FIG. 2. Schematic of the pET-PR-LicKM-KDEL and pET-PR-LicKM-VAC constructs inserted into a modified pET32a vector.

FIG. 3. Schematic of the pBI121 vector organization.

FIG. 4. Schematic organization of the derivation of the pBID4 plasmid from a pBI vector after excision of the GUS gene and the addition of a TMV-derived plasmid.

FIG. 5. Schematic of the fusion of HA, domains of HA, and NA in lichenase sequence, with and without targeting sequences which were put into a vector.

FIG. 6. Lichenase assays of extracts of plants expressing Lic-NA fusion proteins.

FIG. 7. Western analysis of extracts of plants expressing Lic-HA fusion proteins.

FIG. 8. Neuraminidase assays in the presence of anti-NA antibody and a control anti-RSV antibody.

FIG. 9. 2′-(4-Methylumbelliferyl)-α-D-N-acetylneuraminic acid chemical structure.

FIG. 10. Comparison of efficacy of A/Udorn/72 with oseltamivir carboxylate (Tamiflu®) in neuraminidase assays and demonstrating IC₅₀.

FIG. 11. Comparison of efficacy of A/New Caldonia/99 with oseltamivir carboxylate (Tamiflu®)in neuraminidase assays and demonstrating IC₅₀.

FIG. 12. Comparison of efficacy of A/Vietnam/1203/04 with oseltamivir carboxylate (Tamiflu®) in neuraminidase assays and demonstrating IC₅₀.

FIG. 13. Comparison of efficacy of A/Hong Kong/156/97 with oseltamivir carboxylate (Tamiflu®) in neuraminidase assays and demonstrating IC₅₀.

FIG. 14. Comparison of efficacy of A/Indonesia/05 with oseltamivir carboxylate (Tamiflu®) in neuraminidase assays and demonstrating IC₅₀.

### DETAILED DESCRIPTION OF THE INVENTION

The invention relates to influenza antigens useful in the preparation of antibodies against influenza infection, and fusion proteins comprising such influenza antigens operably linked to thermostable protein. The invention relates to antibody compositions, and methods of production of provided antibody compositions, including but not limited to, production in plant systems. Further, the invention relates to vectors, fusion proteins, plant cells, plants and compositions comprising antibodies or antigen binding fragments thereof of the invention. Still further provided are kits as well as therapeutic and diagnostic uses in association with influenza infection in a subject.

### Influenza Antigens

Influenza antigen proteins of the present invention include any immunogenic protein or peptide capable of eliciting an immune response against influenza virus. Generally, immunogenic proteins of interest include influenza antigens (e.g., influenza proteins, fusion proteins, etc.), immunogenic portions thereof, or immunogenic variants thereof and combinations of any of the foregoing.

Influenza antigens for use in accordance with the present invention may include full-length influenza proteins or fragments of influenza proteins, and/or fusion proteins comprising full-length influenza proteins or fragments of influenza proteins. Where fragments of influenza proteins are utilized, whether alone or in fusion proteins, such fragments retain immunological activity (e.g., cross-reactivity with anti-influenza antibodies). Based on their capacity to induce immunoprotective response against viral infection, hemagglutinin and neuraminidase are primary antigens of interest in generating antibodies.

Thus, the invention provides plant cells and plants expressing a heterologous protein (e.g., an influenza antigen, such as an influenza protein or a fragment thereof and/or a fusion protein comprising an influenza protein or fragment thereof). A heterologous protein of the invention can comprise any influenza antigen of interest, including, neuraminidase (NA), a portion of neuraminidase (NA) or fusion proteins, fragments.

Amino acid sequences of a variety of different influenza NA proteins (e.g., from different subtypes, or strains or isolates) are known in the art and are available in public databases such as GenBank. Exemplary full length protein sequences for NA of two influenza subtypes of particular interest today, are provided below:

V: Vietnam H5N1
NA (NAV) SEQ ID NO.: 2:
MNPNQKIITIGSICMVTGIVSLMLQIGNMISIWVSHSIHTGNQHQSEPISNTNLLTEKAVASVKL
AGNSSLCPINGWAVYSKDNSIRIGSKGDVFVIREPFISCSHLECRTFFLTQGALLNDKHSNGT
VKDRSPHRTLMSCPVGEAPSPYNSRFESVAWSASACHDGTSWLTIGISGPDNGAVAVLKYN
GIITDTIKSWRNNILRTQESECACVNGSCFTVMTDGPSNGQASHKIFKMEKGKVVKSVELDA
PNYHYEECSCYPDAGEITCVCRDNWHGSNRPWVSFNQNLEYQIGYICSGVFGDNPRPNDGT
GSCGPVSSNGAGGVKGFSFKYGNGVWIGRTKSTNSRSGFEMIWDPNGWTETDSSFSVKQDI
VAITDWSGYSGSFVQHPELTGLDCIRPCFWVELIRGRPKESTIWTSGSSISFCGVNSDTVGWS
WPDGAELPFTIDK
W: Wyoming H3N2
NA (NAW) SEQ ID NO.: 4:
MNPNQKIITIGSVSLTISTICFFMQIAILITTVTLHFKQYEFNSPPNNQVMLCEPTIIERNITEIVY
LTNTTIEKEICPKLAEYRNWSKPQCNITGFAPFSKDNSIRLSAGGDIWVTREPYVSCDPDKCY
QFALGQGTTLNNVHSNDTVHDRTPYRTLLMNELGVPFHLGTKQVCIAWSSSSCHDGKAWL
HVCVTGDDENATASFIYNGRLVDSIVSWSKKILRTQESECVCINGTCTVVMTDGSASGKAD
TKILFIEEGKIVHTSTLSGSAQHVEECSCYPRYPGVRCVCRDNWKGSNRPIVDINIKDYSIVSS
YVCSGLVGDTPRKNDSSSSSHCLDPNNEEGGHGVKGWAFDDGNDVWMGRTISEKLRSGYE
TFKVIEGWSNPNSKLQINRQVIVDRGNRSGYSGIFSVEGKSCINRCFYVELIRGRKQETEVLW
TSNSIVVFCGTSGTYGTGSWPDGADINLMPI
Influenza Proteins
Neuraminidase
NA Vietnam:
H5N1 NA anchor peptide SEQ ID NO.: 15:
MNPNQKIITIGSICMVTGIVS
H5N1 NA SEQ ID NO.: 16:
LMLQIGNMISIWVSHSIHTGNQHQSEPISNTNLLTEKAVASVKLAGNSSLCPINGWAVYSKD
NSIRIGSKGDVFVIREPFISCSHLECRTFFLTQGALLNDKHSNGTVKDRSPHRTLMSCPVGEA
PSPYNSRFESVAWSASACHDGTSWLTIGISGPDNGAVAVLKYNGIITDTIKSWRNNILRTQES
ECACVNGSCFTVMTDGPSNGQASHKIFKMEKGKVVKSVELDAPNYHYEECSCYPDAGEIT
CVCRDNWHGSNRPWVSFNQNLEYQIGYICSGVFGDNPRPNDGTGSCGPVSSNGAGGVKGF
SFKYGNGVWIGRTKSTNSRSGFEMIWDPNGWTETDSSFSVKQDIVAITDWSGYSGSFVQHP
ELTGLDCIRPCFWVELIRGRPKESTIWTSGSSISFCGVNSDTVGWSWPDGAELPFTIDK
H3N2 NA anchor peptide SEQ ID NO.: 17:
MNPNQKIITIGSVSLTISTICFFMQIAILITTVTLHF
H3N2 NA SEQ ID NO.: 18:
KQYEFNSPPNNQVMLCEPTIIERNITEIVYLTNTTIEKEICPKLAEYRNWSKPQCNITGFAPFS
KDNSIRLSAGGDIWVTREPYVSCDPDKCYQFALGQGTTLNNVHSNDTVHDRTPYRTLLMN
ELGVPFHLGTKQVCIAWSSSSCHDGKAWLHVCVTGDDENATASFIYNGRLVDSIVSWSKKI
LRTQESECVCINGTCTVVMTDGSASGKADTKILFIEEGKIVHTSTLSGSAQHVEECSCYPRYP
GVRCVCRDNWKGSNRPIVDINIKDYSIVSSYVCSGLVGDTPRKNDSSSSSHCLDPNNEEGGH
GVKGWAFDDGNDVWMGRTISEKLRSGYETFKVIEGWSNPNSKLQINRQVIVDRGNRSGYS
GIFSVEGKSCINRCFYVELIRGRKQETEVLWTSNSIVVFCGTSGTYGTGSWPDGADINLMPI

While sequences of exemplary influenza antigens are provided herein, and domains depicted for NA have been provided for exemplary strains, it will be appreciated that any sequence having immunogenic characteristics of a domain of NA may alternatively be employed. One skilled in the art will readily be capable of generating sequences having at least 75%, 80%, 85%, or 90% or more identity to provided antigens. In certain embodiments, influenza antigens comprise proteins including those having at least 95%, 96%, 97%, 98%, or more identity to a domain NA, or a portion of a domain NA, wherein an antigen protein retains immunogenic activity. For example sequences having sufficient identity to influenza antigen(s) which retain immunogenic characteristics are capable of binding with antibodies which react with domains (antigen(s)) provided herein. Immunogenic characteristics often include three dimensional presentation of relevant amino acids or side groups. One skilled in the art can readily identify sequences with modest differences in sequence (e.g., with difference in boundaries and/or some sequence alternatives, that, nonetheless preserve immunogenic characteristics). For instance, sequences whose boundaries are near to (e.g., within about 15 amino acids, 14 amino acids, 13 amino acids, 12 amino acids, 11 amino acids, 10 amino acids, 9 amino acids, 8 amino acids, 7 amino acids 6 amino acids, 5 amino acids 4 amino acids, 3 amino acids, 2 amino acids, or 1 amino acid) of domain boundaries designated herein at either end of designated amino acid sequence may be considered to comprise relevant domain in accordance with the present invention. Thus, the invention contemplates use of a sequence of influenza antigen to comprise residues approximating the domain designation. For example, domain(s) of NA have been engineered and expressed as an in-frame fusion protein as an antigen of the invention (see Examples herein). Further, one will appreciate that any domains, partial domains or regions of amino acid sequence of influenza antigen (e.g., NA) which are immunogenic can be generated using constructs and methods provided herein. Still further, domains or subdomains can be combined, separately and/or consecutively for production of influenza antigens.

As exemplary antigens, we have utilized sequences from neuraminidase, of particular subtypes as described in detail herein. Various subtypes of influenza virus exist and continue to be identified as new subtypes emerge. It will be understood by one skilled in the art that the methods and compositions provided herein may be adapted to utilize sequences of additional subtypes. Such variation is contemplated and encompassed within the methods and compositions provided herein.

Influenza Polypeptide Fusions with Thermostable Proteins

In certain aspects of the invention, provided are influenza antigen(s) comprising fusion polypeptides which comprise an influenza protein (or a fragment or variant thereof) operably linked to a thermostable protein. Inventive fusion polypeptides can be produced in any available expression system known in the art. In certain embodiments, inventive fusion proteins are produced in a plant or portion thereof (e.g., plant, plant cell, root, sprout, etc.).

Enzymes or other proteins which are not found naturally in humans or animal cells are particularly appropriate for use in fusion polypeptides of the present invention. Thermostable proteins that, when fused, confer thermostability to a fusion product are useful. Thermostability allows produced protein to maintain conformation, and maintain produced protein at room temperature. This feature facilitates easy, time efficient and cost effective recovery of a fusion polypeptide. A representative family of thermostable enzymes useful in accordance with the invention is the glucanohydrolase family. These enzymes specifically cleave 1,4-β glucosidic bonds that are adjacent to 1,3-β linkages in mixed linked polysaccharides (Hahn et al., 1994, Proc. Natl. Acad. Sci., USA, 91:10417). Such enzymes are found in cereals, such as oat and barley, and are also found in a number of fungal and bacterial species, including C. thermocellum (Goldenkova et al., 2002, Mol. Biol., 36:698). Thus, desirable thermostable proteins for use in fusion polypeptides of the present invention include glycosidase enzymes. Exemplary thermostable glycosidase proteins include those represented by GenBank accession numbers selected from those set forth in Table A, the contents of each of which are incorporated herein by reference by entire incorporation of the GenBank accession information for each referenced number. Exemplary thermostable enzymes of use in fusion proteins of the invention include Clostridium thermocellum P29716, Brevibacillus brevis P37073, and Rhodthermus marinus P45798, each of which are incorporated herein by reference to their GenBank accession numbers. Representative fusion proteins illustrated in the Examples utilize modified thermostable enzyme isolated from Clostridium thermocellum, however, any thermostable protein may be similarly utilized in accordance with the present invention.

TABLE A
Thermostable glycosidase proteins

| | |
|---|---|
| P29716 | (Beta-glucanase Clostridium thermocellum) |
| P37073 | (Beta-glucanase Brevibacillus brevis) |
| 1MVE_A | (Beta-glucanase Fibrobacter succinogenes) |
| P07883 | (Extracellular agarase Streptomyces coelicolor) |
| P23903 | (Glucan endo-13-beta-glucosidase A1 Bacillus circulans) |
| P27051 | (Beta-glucanase Bacillus licheniformis) |
| P45797 | (Beta-glucanase Paenibacillus polymyxa (Bacillus |
| | polymyxa)) |
| P37073 | (Beta-glucanase Brevibacillus brevis) |
| P45798 | (Beta-glucanase Rhodothermus marinus) |
| P38645 | (Beta-glucosidase Thermobispora bispora) |
| P40942 | (Celloxylanase Clostridium stercorarium) |
| P14002 | (Beta-glucosidase Clostridium thermocellum) |
| O33830 | (Alpha-glucosidase Thermotoga maritima) |
| O43097 | (Xylanase Thermomyces lanuginosus) |
| P54583 | (Endo-glucanase E1 Acidothermus cellulolyticus) |
| P14288 | (Beta-galactosidase Sulfolobus acidocaldarius) |
| O52629 | (Beta-galactosidase Pyrococcus woesei) |
| P29094 | (Oligo-16-glucosidase Geobacillus thermoglucosidasius) |
| P49067 | (Alpha-amylase Pyrococcus furiosus) |
| JC7532 | (Cellulase Bacillus species) |
| Q60037 | (Xylanase A Thermotoga maritima) |
| P33558 | (Xylanase A Clostridium stercorarium) |
| P05117 | (Polygalacturonase-2 precursor Solanum lycopersicum) |
| P04954 | (Cellulase D Clostridium thermocellum) |
| Q4J929 | (N-glycosylase Sulfolobus acidocaldarius) |
| O33833 | (Beta-fructosidase Thermotoga maritima) |
| P49425 | (Endo-14-beta-mannosidase Rhodothermus marinus) |
| P06279 | (Alpha-amylase Geobacillus stearothermophilus) |
| P45702 | (Xylanase Geobacillus stearothermophilus) |
| P45703 | |
| P40943 | |
| P09961 | (Alpha-amylase 1 Dictyoglomus thermophilum) |
| Q60042 | (Xylanase A Thermotoga neapolitana) |
| AAN05438 | (Beta-glycosidase Thermus thermophilus) |
| AAN05439 | |
| AAN05437 | (Sugar permease Thermus thermophilus) |
| AAN05440 | (Beta-glycosidase Thermus filiformis) |
| AAD43138 | (Beta-glycosidase Thermosphaera aggregans) |

When designing fusion proteins and polypeptides in accordance with the invention, it is desirable, of course, to preserve immunogenicity of the antigen. Still further, it is desirable in certain aspects of the invention to provide constructs which provide thermostability of a fusion protein. This feature facilitates easy, time efficient and cost effective recovery of a target antigen. In certain aspects, antigen fusion partners may be selected which provide additional advantages, including enhancement of immunogenicity, potential to incorporate multiple antigenic determinants, yet lack prior immunogenic exposure to vaccination subjects. Further beneficial qualities of fusion peptides of interest include proteins which provide ease of manipulation for incorporation of one or more antigens, as well as proteins which have potential to confer ease of production, purification, and/or formulation for antigen and/or antibody preparations. One of ordinary skill in the art will appreciate that three dimensional presentation can affect each of these beneficial characteristics. Preservation of immunity or preferential qualities therefore may affect, for example, choice of fusion partner and/or choice of fusion location (e.g., N-terminus, C-terminus, internal, combinations thereof). Alternatively or additionally, preferences may affects length of segment selected for fusion, whether it is length of antigen, or length of fusion partner selected.

The present inventors have demonstrated successful fusion of a variety of antigens with a thermostable protein. For example, we have used the thermostable carrier molecule LicB, also referred to as lichenase, for production of fusion proteins. LicB is 1,3-1,4-β glucanase (LicB) from Clostridium thermocellum (GenBank accession: X63355 [gi:40697]). LicB belongs to a family of globular proteins. Based on the three dimensional structure of LicB, its N— and C-termini are situated close to each other on the surface, in close proximity to the active domain. LicB also has a loop structure exposed on the surface that is located far from the active domain. We have generated constructs such that the loop structure and N—0 and C-termini of protein can be used as insertion sites for influenza antigen polypeptides. Influenza antigen polypeptides can be expressed as N— or C-terminal fusions or as inserts into the surface loop. Importantly, LicB maintains its enzymatic activity at low pH and at high temperature (up to 75° C.). Thus, use of LicB as a carrier molecule contributes advantages, including likely enhancement of target specific immunogenicity, potential to incorporate multiple antigen determinants, and straightforward formulation of antigen and/or antibody that may be delivered nasally, orally or parenterally. Furthermore, production of LicB fusions in plants should reduce the risk of contamination with animal or human pathogens. See examples provided herein.

Fusion proteins of the invention comprising influenza antigen may be produced in any of a variety of expression systems, including both in vitro and in vivo systems. One skilled in the art will readily appreciate that optimization of nucleic acid sequences for a particular expression system is often desirable. For example, in the exemplification provided herein, optimized sequence for expression of influenza antigen-LicB fusions in plants is provided (Example 1). Thus, any relevant nucleic acid encoding influenza antigen(s) fusion protein(s) and fragments thereof in accordance with the invention is intended to be encompassed within nucleic acid constructs of the invention.

For production in plant systems, transgenic plants expressing influenza antigen(s) (e.g., influenza protein(s) or fragments or fusions thereof) may be utilized. Alternatively or additionally, transgenic plants may be produced using methods well known in the art to generate stable production crops. Additionally, plants utilizing transient expression systems may be utilized for production of influenza antigen(s). When utilizing plant expression systems, whether transgenic or transient expression in plants is utilized, any of nuclear expression, chloroplast expression, mitochondrial expression, or viral expression may be taken advantage of according to the applicability of the system to antigen desired. Furthermore, additional expression systems for production of antigens and fusion proteins in accordance with the present invention may be utilized. For example, mammalian expression systems (e.g., mammalian cell lines, such as CHO, etc.), bacterial expression systems (e.g., E. coli), insect expression systems (e.g., baculovirus), yeast expression systems, and in vitro expression systems (e.g., reticulate lysates) may be used for expression of antigens and fusion proteins of the invention.

### Production of Influenza Antigens

In accordance with the present invention, influenza antigens (including influenza protein(s), fragments, variants, and/or fusions thereof) may be produced in any desirable system; production is not limited to plant systems. Vector constructs and expression systems are well known in the art and may be adapted to incorporate use of influenza antigens provided herein. For example, influenza antigens (including fragments, variants, and/or fusions) can be produced in known expression systems, including mammalian cell systems, transgenic animals, microbial expression systems, insect cell systems, and plant systems, including transgenic and transient plant systems. Particularly where influenza antigens are produced as fusion proteins, it may be desirable to produce such fusion proteins in non-plant systems.

In some embodiments of the invention, influenza antigens are desirably produced in plant systems. Plants are relatively easy to manipulate genetically, and have several advantages over alternative sources such as human fluids, animal cell lines, recombinant microorganisms and transgenic animals. Plants have sophisticated post-translational modification machinery for proteins that is similar to that of mammals (although it should be noted that there are some differences in glycosylation patterns between plants and mammals). This enables production of bioactive reagents in plant tissues. Also, plants can economically produce very large amounts of biomass without requiring sophisticated facilities. Moreover, plants are not subject to contamination with animal pathogens. Like liposomes and microcapsules, plant cells are expected to provide protection for passage of antigen to the gastrointestinal tract.

Plants may be utilized for production of heterologous proteins via use of various production systems. One such system includes use of transgenic/genetically-modified plants where a gene encoding target product is permanently incorporated into the genome of the plant. Transgenic systems may generate crop production systems. A variety of foreign proteins, including many of mammalian origin and many vaccine candidate antigens, have been expressed in transgenic plants and shown to have functional activity (Tacket et al., 2000, J. Infect. Dis., 182:302; and Thanavala et al., 2005, Proc. Natl. Acad. Sci., USA, 102:3378). Additionally, administration of unprocessed transgenic plants expressing hepatitis B major surface antigen to non-immunized human volunteers resulted in production of immune response (Kapusta et al., 1999, FASEB J., 13:1796).

Another system for expressing polypeptides in plants utilizes plant viral vectors engineered to express foreign sequences (e.g., transient expression). This approach allows for use of healthy non-transgenic plants as rapid production systems. Thus, genetically engineered plants and plants infected with recombinant plant viruses can serve as “green factories” to rapidly generate and produce specific proteins of interest. Plant viruses have certain advantages that make them attractive as expression vectors for foreign protein production. Several members of plant RNA viruses have been well characterized, and infectious cDNA clones are available to facilitate genetic manipulation. Once infectious viral genetic material enters a susceptible host cell, it replicates to high levels and spreads rapidly throughout the entire plant. There are several approaches to producing target polypeptides using plant viral expression vectors, including incorporation of target polypeptides into viral genomes. One approach involves engineering coat proteins of viruses that infect bacteria, animals or plants to function as carrier molecules for antigenic peptides. Such carrier proteins have the potential to assemble and form recombinant virus-like particles displaying desired antigenic epitopes on their surface. This approach allows for time-efficient production of antigen and/or antibody candidates, since the particulate nature of an antigen and/or antibody candidate facilitates easy and cost-effective recovery from plant tissue. Additional advantages include enhanced target-specific immunogenicity, the potential to incorporate multiple antigen determinants and/or antibody sequences, and ease of formulation into antigen and/or antibody that can be delivered nasally, orally or parenterally. As an example, spinach leaves containing recombinant plant viral particles carrying epitopes of virus fused to coat protein have generated immune response upon administration (Modelska et al., 1998, Proc. Natl. Acad. Sci., USA, 95:2481; and Yusibov et al., 2002, Vaccine, 19/20:3155).

### Plant Expression Systems

Any plant susceptible to incorporation and/or maintenance of heterologous nucleic acid and capable of producing heterologous protein may be utilized in accordance with the present invention. In general, it will often be desirable to utilize plants that are amenable to growth under defined conditions, for example in a greenhouse and/or in aqueous systems. It may be desirable to select plants that are not typically consumed by human beings or domesticated animals and/or are not typically part of the human food chain, so that they may be grown outside without concern that expressed polynucleotide may be undesirably ingested. In some embodiments, however, it will be desirable to employ edible plants. In particular embodiments, it will be desirable to utilize plants that accumulate expressed polypeptides in edible portions of the plant.

Often, certain desirable plant characteristics will be determined by the particular polynucleotide to be expressed. To give but a few examples, when a polynucleotide encodes a protein to be produced in high yield (as will often be the case, for example, when antigen proteins are to be expressed), it will often be desirable to select plants with relatively high biomass (e.g., tobacco, which has additional advantages that it is highly susceptible to viral infection, has a short growth period, and is not in the human food chain). Where a polynucleotide encodes antigen protein whose full activity requires (or is inhibited by) a particular post-translational modification, the ability (or inability) of certain plant species to accomplish relevant modification (e.g., a particular glycosylation) may direct selection. For example, plants are capable of accomplishing certain post-translational modifications (e.g., glycosylation); however, plants will not generate sialation patterns which are found in mammalian post-translational modification. Thus, plant production of antigen may result in production of a different entity than the identical protein sequence produced in alternative systems.

In certain embodiments of the invention, crop plants, or crop-related plants are utilized. In certain specific embodiments, edible plants are utilized.

Plants for use in accordance with the present invention include Angiosperms, Bryophytes (e.g., Hepaticae, Musci, etc.), Pteridophytes (e.g., ferns, horsetails, lycopods), Gymnosperms (e.g., conifers, cycase, Ginko, Gnetales), and Algae (e.g., Chlorophyceae, Phaeophyceae, Rhodophyceae, Myxophyceae, Xanthophyceae, and Euglenophyceae). Exemplary plants are members of the family Leguminosae (Fabaceae; e.g., pea, alfalfa, soybean); Gramineae (Poaceae; e.g., corn, wheat, rice); Solanaceae, particularly of the genus Lycopersicon (e.g., tomato), Solanum (e.g., potato, eggplant), Capsium (e.e., pepper), or Nicotiana (e.g., tobacco); Umbelliferae, particularly of the genus Daucus (e.g., carrot), Apium (e.g., celery), or Rutaceae (e.g., oranges); Compositae, particularly of the genus Lactuca (e.g., lettuce); Brassicaceae (Cruciferae), particularly of the genus Brassica or Sinapis. In certain aspects, exemplary plants of the invention may be plants of the Brassica or Arabidopsis genus. Some exemplary Brassicaceae family members include Brassica campestris, B. carinata, B. juncea, B. napus, B. nigra, B. oleraceae, B. tournifortii, Sinapis alba, and Raphanus sativus. Some suitable plants that are amendable to transformation and are edible as sprouted seedlings include alfalfa, mung bean, radish, wheat, mustard, spinach, carrot, beet, onion, garlic, celery, rhubarb, a leafy plant such as cabbage or lettuce, watercress or cress, herbs such as parsley, mint, or clovers, cauliflower, broccoli, soybean, lentils, edible flowers such as sunflower etc.

Introducing Vectors into Plants

In general, vectors may be delivered to plants according to known techniques. For example, vectors themselves may be directly applied to plants (e.g., via abrasive inoculations, mechanized spray inoculations, vacuum infiltration, particle bombardment, or electroporation). Alternatively or additionally, virions may be prepared (e.g., from already infected plants), and may be applied to other plants according to known techniques.

A wide variety of viruses are known that infect various plant species, and can be employed for polynucleotide expression according to the present invention (see, for example, The Classification and Nomenclature of Viruses, “Sixth Report of the International Committee on Taxonomy of Viruses,” Ed. Murphy et al., Springer Verlag: New York, 1995, the entire contents of which are incorporated herein by reference; Grierson et al., Plant Molecular Biology, Blackie, London, pp. 126-146, 1984; Gluzman et al., Communications in Molecular Biology: Viral Vectors, Cold Spring Harbor Laboratory, Cold Spring Harbor, N.Y., pp. 172-189, 1988; and Mathew, Plant Viruses Online, http://image.fs.uidaho.edu/vide/). In certain embodiments of the invention rather than delivering a single viral vector to a plant cell, multiple different vectors are delivered which, together, allow for replication (and, optionally cell-to-cell and/or long distance movement) of viral vector(s). Some or all of the proteins may be encoded by the genome of transgenic plants. In certain aspects, described in further detail herein, these systems include one or more viral vector components.

Vector systems that include components of two heterologous plant viruses in order to achieve a system that readily infects a wide range of plant types and yet poses little or no risk of infectious spread. An exemplary system has been described previously (see, e.g., PCT Publication WO 00/25574 and U.S. Patent Publication 2005/0026291, which is incorporated herein by reference). As noted herein, in particular aspects of the present invention, viral vectors are applied to plants (e.g., plant, portion of plant, sprout, etc.) by various methods (e.g., through infiltration or mechanical inoculation, spray, etc.). Where infection is to be accomplished by direct application of a viral genome to a plant, any available technique may be used to prepare the genome. For example, many viruses that are usefully employed in accordance with the present invention have ssRNA genomes. ssRNA may be prepared by transcription of a DNA copy of the genome, or by replication of an RNA copy, either in vivo or in vitro. Given the readily availability of easy-to-use in vitro transcription systems (e.g., SP6, T7, reticulocyte lysate, etc.), and also the convenience of maintaining a DNA copy of an RNA vector, it is expected that inventive ssRNA vectors will often be prepared by in vitro transcription, particularly with T7 or SP6 polymerase.

In certain embodiments of the invention rather than introducing a single viral vector type into a plant, multiple different viral vectors are introduced. Such vectors may, for example, trans-complement each other with respect to functions such as replication, cell-to-cell movement, and/or long distance movement. Vectors may contain different polynucleotides encoding influenza antigen of the invention. Selection for plant(s) or portions thereof that express multiple polypeptides encoding one or more influenza antigen(s) may be performed as described above for single polynucleotides or polypeptides.

Plant Tissue Expression Systems

As discussed above, in accordance with the present invention, influenza antigens may be produced in any desirable system. Vector constructs and expression systems are well known in the art and may be adapted to incorporate use of influenza antigens provided herein. For example, transgenic plant production is known and generation of constructs and plant production may be adapted according to known techniques in the art. In some embodiments, transient expression systems in plants are desired. Two of these systems include production of clonal roots and clonal plant systems, and derivatives thereof, as well as production of sprouted seedlings systems.

Clonal Plants

Clonal roots maintain RNA viral expression vectors and stably produce target protein uniformly in the entire root over extended periods of time and multiple subcultures. In contrast to plants, where a target gene is eliminated via recombination during cell-to-cell or long distance movement, in root cultures the integrity of a viral vector is maintained and levels of target protein produced over time are similar to those observed during initial screening. Clonal roots allow for ease of production of heterologous protein material for oral formulation of antigen and antibody compositions. Methods and reagents for generating a variety of clonal entities derived from plants which are useful for the production of antigen (e.g., antigen proteins of the invention) have been described previously and are known in the art (see, for example, PCT Publication WO 05/81905, which is incorporated herein by reference). Clonal entities include clonal root lines, clonal root cell lines, clonal plant cell lines, and clonal plants capable of production of antigen (e.g., antigen proteins of the invention). The invention further provides methods and reagents for expression of antigen polynucleotide and polypeptide products in clonal cell lines derived from various plant tissues (e.g., roots, leaves), and in whole plants derived from single cells (clonal plants). Such methods are typically based on use of plant viral vectors of various types.

For example, in one aspect, the invention provides methods of obtaining a clonal root line that expresses a polynucleotide encoding an influenza antigen of the invention comprising steps of: (i) introducing a viral vector that comprises a polynucleotide encoding an influenza antigen of the invention into a plant or portion thereof; and (ii) generating one or more clonal root lines from a plant. Clonal root lines may be generated, for example, by infecting a plant or plant portion (e.g., a harvested piece of leaf) with an Agrobacterium (e.g., A. rhizogenes) that causes formation of hairy roots. Clonal root lines can be screened in various ways to identify lines that maintain virus, lines that express a polynucleotide encoding an influenza antigen of the invention at high levels, etc. The invention further provides clonal root lines, e.g., clonal root lines produced according to inventive methods and further encompasses methods of expressing polynucleotides and producing polypeptide(s) encoding influenza antigen(s) of the invention using clonal root lines.

The invention further provides methods of generating a clonal root cell line that expresses a polynucleotide encoding an influenza antigen of the invention comprising steps of: (i) generating a clonal root line, cells of which contain a viral vector whose genome comprises a polynucleotide encoding an influenza antigen of the invention; (ii) releasing individual cells from a clonal root line; and (iii) maintaining cells under conditions suitable for root cell proliferation. The invention provides clonal root cell lines and methods of expressing polynucleotides and producing polypeptides using clonal root cell lines.

In one aspect, the invention provides methods of generating a clonal plant cell line that expresses a polynucleotide encoding an influenza antigen of the invention comprising steps of: (i) generating a clonal root line, cells of which contain a viral vector whose genome comprises a polynucleotide encoding an influenza antigen of the invention; (ii) releasing individual cells from a clonal root line; and (iii) maintaining cells in culture under conditions appropriate for plant cell proliferation. The invention further provides methods of generating a clonal plant cell line that expresses a polynucleotide encoding an influenza antigen of the invention comprising steps of: (i) introducing a viral vector that comprises a polynucleotide encoding an influenza antigen of the invention into cells of a plant cell line maintained in culture; and (ii) enriching for cells that contain viral vector. Enrichment may be performed, for example, by (i) removing a portion of cells from the culture; (ii) diluting removed cells so as to reduce cell concentration; (iii) allowing diluted cells to proliferate; and (iv) screening for cells that contain viral vector. Clonal plant cell lines may be used for production of an influenza antigen in accordance with the present invention.

The invention includes a number of methods for generating clonal plants, cells of which contain a viral vector that comprises a polynucleotide encoding influenza antigen of the invention. For example, the invention provides methods of generating a clonal plant that expresses a polynucleotide encoding influenza antigen of the invention comprising steps of: (i) generating a clonal root line, cells of which contain a viral vector whose genome comprises a polynucleotide encoding influenza antigen of the invention; (ii) releasing individual cells from a clonal root line; and (iii) maintaining released cells under conditions appropriate for formation of a plant. The invention further provides methods of generating a clonal plant that expresses a polynucleotide encoding influenza antigen of the invention comprising steps of: (i) generating a clonal plant cell line, cells of which contain a viral vector whose genome comprises a polynucleotide encoding an influenza antigen of the invention; and (ii) maintaining cells under conditions appropriate for formation of a plant. In general, clonal plants according to the invention can express any polynucleotide encoding an influenza antigen of the invention. Such clonal plants can be used for production of an antigen polypeptide.

As noted above, the present invention provides systems for expressing a polynucleotide or polynucleotide(s) encoding influenza antigen(s) of the invention in clonal root lines, clonal root cell lines, clonal plant cell lines (e.g., cell lines derived from leaf, stem, etc.), and in clonal plants. A polynucleotide encoding an influenza antigen of the invention is introduced into an ancestral plant cell using a plant viral vector whose genome includes polynucleotide encoding an influenza antigen of the invention operably linked to (i.e., under control of) a promoter. A clonal root line or clonal plant cell line is established from a cell containing virus according to any of several techniques further described below. The plant virus vector or portions thereof can be introduced into a plant cell by infection, by inoculation with a viral transcript or infectious cDNA clone, by electroporation, by T-DNA mediated gene transfer, etc.

The following sections describe methods for generating clonal root lines, clonal root cell lines, clonal plant cell lines, and clonal plants that express a polynucleotide encoding an influenza antigen of the invention are then described. A “root line” is distinguished from a “root cell line” in that a root line produces actual root-like structures or roots while a root cell line consists of root cells that do not form root-like structures. Use of the term “line” is intended to indicate that cells of the line can proliferate and pass genetic information on to progeny cells. Cells of a cell line typically proliferate in culture without being part of an organized structure such as those found in an intact plant. Use of the term “root line” is intended to indicate that cells in the root structure can proliferate without being part of a complete plant. It is noted that the term “plant cell” encompasses root cells. However, to distinguish the inventive methods for generating root lines and root cell lines from those used to directly generate plant cell lines from non-root tissue (as opposed to generating clonal plant cell lines from clonal root lines or clonal plants derived from clonal root lines), the terms “plant cell” and “plant cell line” as used herein generally refer to cells and cell lines that consist of non-root plant tissue. Plant cells can be, for example, leaf, stem, shoot, flower part, etc. It is noted that seeds can be derived from clonal plants generated as derived herein. Such seeds may contain viral vector as will plants obtained from such seeds. Methods for obtaining seed stocks are well known in the art (see, e.g., U.S. Patent Publication 2004/0093643).

Clonal Root Lines

The present invention provides systems for generating a clonal root line in which a plant viral vector is used to direct expression of a polynucleotide encoding an influenza antigen of the invention. One or more viral expression vector(s) including a polynucleotide encoding an influenza antigen of the invention operably linked to a promoter is introduced into a plant or a portion thereof according to any of a variety of known methods. For example, plant leaves can be inoculated with viral transcripts. Vectors themselves may be directly applied to plants (e.g., via abrasive inoculations, mechanized spray inoculations, vacuum infiltration, particle bombardment, or electroporation). Alternatively or additionally, virions may be prepared (e.g., from already infected plants), and may be applied to other plants according to known techniques.

Where infection is to be accomplished by direct application of a viral genome to a plant, any available technique may be used to prepare viral genome. For example, many viruses that are usefully employed in accordance with the present invention have ssRNA genomes. ssRNA may be prepared by transcription of a DNA copy of the genome, or by replication of an RNA copy, either in vivo or in vitro. Given the readily available, easy-to-use in vitro transcription systems (e.g., SP6, T7, reticulocyte lysate, etc.), and also the convenience of maintaining a DNA copy of an RNA vector, it is expected that inventive ssRNA vectors will often be prepared by in vitro transcription, particularly with T7 or SP6 polymerase. Infectious cDNA clones can be used. Agrobacterially mediated gene transfer can be used to transfer viral nucleic acids such as viral vectors (either entire viral genomes or portions thereof) to plant cells using, e.g., agroinfiltration, according to methods known in the art.

A plant or plant portion may then be then maintained (e.g., cultured or grown) under conditions suitable for replication of viral transcript. In certain embodiments of the invention virus spreads beyond the initially inoculated cell, e.g., locally from cell to cell and/or systemically from an initially inoculated leaf into additional leaves. However, in some embodiments of the invention virus does not spread. Thus viral vector may contain genes encoding functional MP and/or CP, but may be lacking one or both of such genes. In general, viral vector is introduced into (infects) multiple cells in the plant or portion thereof.

Following introduction of viral vector into a plant, leaves are harvested. In general, leaves may be harvested at any time following introduction of viral vector. However, it may be desirable to maintain the plant for a period of time following introduction of viral vector into a plant, e.g., a period of time sufficient for viral replication and, optionally, spread of virus from cells into which it was initially introduced. A clonal root culture (or multiple cultures) is prepared, e.g., by known methods further described below.

In general, any available method may be used to prepare a clonal root culture from a plant or plant tissue into which a viral vector has been introduced. One such method employs genes that exist in certain bacterial plasmids. These plasmids are found in various species of Agrobacterium that infect and transfer DNA to a wide variety of organisms. As a genus, Agrobacteria can transfer DNA to a large and diverse set of plant types including numerous dicot and monocot angiosperm species and gymnosperms (see Gelvin et al., 2003, Microbiol. Mol. Biol. Rev., 67:16) and references therein, all of which are incorporated herein by reference). The molecular basis of genetic transformation of plant cells is transfer from bacterium and integration into plant nuclear genome of a region of a large tumor-inducing (Ti) or rhizogenic (Ri) plasmid that resides within various Agrobacterial species. This region is referred to as the T-region when present in the plasmid and as T-DNA when excised from plasmid. Generally, a single-stranded T-DNA molecule is transferred to a plant cell in naturally occurring Agrobacterial infection and is ultimately incorporated (in double-stranded form) into the genome. Systems based on Ti plasmids are widely used for introduction of foreign genetic material into plants and for production of transgenic plants.

Infection of plants with various Agrobacterial species and transfer of T-DNA has a number of effects. For example, A. tumefaciens causes crown gall disease while A. rhizogenes causes development of hairy roots at the site of infection, a condition known as “hairy root disease.” Each root arises from a single genetically transformed cell. Thus root cells in roots are clonal, and each root represents a clonal population of cells. Roots produced by A. rhizogenes infection are characterized by a high growth rate and genetic stability (Giri et al., 2000, Biotechnol. Adv., 18:1, and references therein, all of which are incorporated herein by reference). In addition, such roots are able to regenerate genetically stable plants (Giri et al., 2000, supra).

In general, the present invention encompasses use of any strain of Agrobacteria, (e.g., any A. rhizogenes strain) that is capable of inducing formation of roots from plant cells. As mentioned above, a portion of the Ri plasmid (Ri T-DNA) is responsible for causing hairy root disease. While transfer of this portion of the Ri plasmid to plant cells can conveniently be accomplished by infection with Agrobacteria harboring the Ri plasmid, the invention encompasses use of alternative methods of introducing the relevant region into a plant cell. Such methods include any available method of introducing genetic material into plant cells including, but not limited to, biolistics, electroporation, PEG-mediated DNA uptake, Ti-based vectors, etc. The relevant portions of Ri T-DNA can be introduced into plant cells by use of a viral vector. Ri genes can be included in the same vector that contains a polynucleotide encoding an influenza antigen of the invention or in a different viral vector, which can be the same or a different type to that of the vector that contains a polynucleotide encoding an influenza antigen of the invention. It is noted that the entire Ri T-DNA may not be required for production of hairy roots, and the invention encompasses use of portions of Ri T-DNA, provided that such portions contain sufficient genetic material to induce root formation, as known in the art. Additional genetic material, e.g., genes present within the Ri plasmid but not within T-DNA, may be transferred to a plant cell in accordance with the invention, particularly genes whose expression products facilitate integration of T-DNA into the plant cell DNA.

In order to prepare a clonal root line in accordance with certain embodiments of the invention, harvested leaf portions are contacted with A. rhizogenes under conditions suitable for infection and transformation. Leaf portions are maintained in culture to allow development of hairy roots. Each root is clonal, i.e., cells in the root are derived from a single ancestral cell into which Ri T-DNA was transferred. In accordance with the invention, a portion of such ancestral cells may contain viral vector. Thus cells in a root derived from such an ancestral cell may contain viral vector since it will be replicated and will be transmitted during cell division. Thus a high proportion (e.g., at least 50%, at least 75%, at least 80%, at least 90%, at least 95%), all (100%), or substantially all (at least 98%) of cells will contain viral vector. It is noted that since viral vector is inherited by daughter cells within a clonal root, movement of viral vector within the root is not necessary to maintain viral vector throughout the root. Individual clonal hairy roots may be removed from the leaf portion and further cultured. Such roots are also referred to herein as root lines. Isolated clonal roots continue to grow following isolation.

A variety of different clonal root lines have been generated using inventive methods. These root lines were generated using viral vectors containing polynucleotide(s) encoding an influenza antigen of the invention (e.g., encoding influenza polypeptide(s), or fragments or fusion proteins thereof). Root lines were tested by Western blot. Root lines displayed a variety of different expression levels of various polypeptides. Root lines displaying high expression were selected and further cultured. These root lines were subsequently tested again and shown to maintain high levels of expression over extended periods of time, indicating stability. Expression levels were comparable to or greater than expression in intact plants infected with the same viral vector used to generate clonal root lines. In addition, stability of expression of root lines was superior to that obtained in plants infected with the same viral vector. Up to 80% of such virus-infected plants reverted to wild type after 2-3 passages. (Such passages involved inoculating plants with transcripts, allowing infection (local or systemic) to become established, taking a leaf sample, and inoculating fresh plants that are subsequently tested for expression.)

Root lines may be cultured on a large scale for production of antigen of the invention polypeptides as discussed further below. It is noted that clonal root lines (and cell lines derived from clonal root lines) can generally be maintained in medium that does not include various compounds, e.g., plant growth hormones such as auxins, cytokinins, etc., that are typically employed in culture of root and plant cells. This feature greatly reduces expense associated with tissue culture, and the inventors expect that it will contribute significantly to economic feasibility of protein production using plants.

Any of a variety of methods may be used to select clonal roots that express a polynucleotide encoding influenza antigen(s) of the invention. Western blots, ELISA assays, etc., can be used to detect an encoded polypeptide. In the case of detectable markers such as GFP, alternative methods such as visual screens can be performed. If a viral vector that contains a polynucleotide that encodes a selectable marker is used, an appropriate selection can be imposed (e.g., leaf material and/or roots derived therefrom can be cultured in the presence of an appropriate antibiotic or nutritional condition and surviving roots identified and isolated). Certain viral vectors contain two or more polynucleotide(s) encoding influenza antigen(s) of the invention, e.g., two or more polynucleotides encoding different polypeptides. If one of these is a selectable or detectable marker, clonal roots that are selected or detected by selecting for or detecting expression of the marker will have a high probability of also expressing a second polynucleotide. Screening for root lines that contain particular polynucleotides can also be performed using PCR and other nucleic acid detection methods.

Alternatively or additionally, clonal root lines can be screened for presence of virus by inoculating host plants that will form local lesions as a result of virus infection (e.g., hypersensitive host plants). For example, 5 mg of root tissue can be homogenized in 50 μl of phosphate buffer and used to inoculate a single leaf of a tobacco plant. If virus is present in root cultures, within two to three days characteristic lesions will appear on infected leaves. This means that root line contains recombinant virus that carries a polynucleotide encoding an influenza antigen of the invention (a target gene). If no local lesions are formed, there is no virus, and the root line is rejected as negative. This method is highly time- and cost-efficient. After initially screening for the presence of virus, roots that contain virus may be subjected to secondary screening, e.g., by Western blot or ELISA to select high expressers. Additional screens, e.g., screens for rapid growth, growth in particular media or under particular environmental conditions, etc., can be applied. These screening methods may, in general, be applied in the development of any of clonal root lines, clonal root cell lines, clonal plant cell lines, and/or clonal plants described herein.

As will be evident to one of ordinary skill in the art, a variety of modifications may be made to the description of the inventive methods for generating clonal root lines that contain a viral vector. Such modifications are within the scope of the invention. For example, while it is generally desirable to introduce viral vector into an intact plant or portion thereof prior to introduction of Ri T-DNA genes, in certain embodiments of the invention the Ri-DNA is introduced prior to introducing viral vector. In addition, it is possible to contact intact plants with A. rhizogenes rather than harvesting leaf portions and then exposing them to bacterium.

Other methods of generating clonal root lines from single cells of a plant or portion thereof that harbor a viral vector can be used (i.e., methods not using A. rhizogenes or genetic material from the Ri plasmid). For example, treatment with certain plant hormones or combinations of plant hormones is known to result in generation of roots from plant tissue.

Clonal Cell Lines Derived from Clonal Root Lines

As described above, the invention provides methods for generating clonal root lines, wherein cells in root lines contain a viral vector. As is well known in the art, a variety of different cell lines can be generated from roots. For example, root cell lines can be generated from individual root cells obtained from a root using a variety of known methods. Such root cell lines may be obtained from various different root cell types within the root. In general, root material is harvested and dissociated (e.g., physically and/or enzymatically digested) to release individual root cells, which are then further cultured. Complete protoplast formation is generally not necessary. If desired, root cells can be plated at very dilute cell concentrations, so as to obtain root cell lines from single root cells. Root cell lines derived in this manner are clonal root cell lines containing viral vector. Such root cell lines therefore exhibit stable expression of a polynucleotide encoding an influenza antigen of the invention. Clonal plant cell lines can be obtained in a similar manner from clonal roots, e.g., by culturing dissociated root cells in the presence of appropriate plant hormones. Screens and successive rounds of enrichment can be used to identify cell lines that express a polynucleotide encoding an influenza antigen of the invention at high levels. However, if the clonal root line from which the cell line is derived already expresses at high levels, such additional screens may be unnecessary.

As in the case of clonal root lines, cells of a clonal root cell line are derived from a single ancestral cell that contains viral vector and may, therefore, contain viral vector since it will be replicated and will be transmitted during cell division. Thus a high proportion(e.g., at least 50%, at least 75%, at least 80%, at least 90%, at least 95%), all (100%), or substantially all (at least 98%) of cells will contain viral vector. It is noted that since viral vector is inherited by daughter cells within a clonal root cell line, movement of viral vector among cells is not necessary to maintain viral vector. Clonal root cell lines can be used for production of a polynucleotide encoding influenza antigen of the invention as described below.

Clonal Plant Cell Lines

The present invention provides methods for generating a clonal plant cell line in which a plant viral vector is used to direct expression of a polynucleotide encoding an influenza antigen of the invention. According to the inventive method, one or more viral expression vector(s) including a polynucleotide encoding an influenza antigen of the invention operably linked to a promoter is introduced into cells of a plant cell line that is maintained in cell culture. A number of plant cell lines from various plant types are known in the art, any of which can be used. Newly derived cell lines can be generated according to known methods for use in practicing the invention. A viral vector is introduced into cells of a plant cell line according to any of a number of methods. For example, protoplasts can be made and viral transcripts then electroporated into cells. Other methods of introducing a plant viral vector into cells of a plant cell line can be used.

A method for generating clonal plant cell lines in accordance with the invention and a viral vector suitable for introduction into plant cells (e.g., protoplasts) can be used as follows: Following introduction of viral vector, a plant cell line may be maintained in tissue culture. During this time viral vector may replicate, and polynucleotide(s) encoding an influenza antigen(s) of the invention may be expressed. Clonal plant cell lines are derived from culture, e.g., by a process of successive enrichment. For example, samples may be removed from culture, optionally with dilution so that the concentration of cells is low, and plated in Petri dishes in individual droplets. Droplets are then maintained to allow cell division.

It will be appreciated that droplets may contain a variable number of cells, depending on the initial density of the culture and the amount of dilution. Cells can be diluted such that most droplets contain either 0 or 1 cell if it is desired to obtain clonal cell lines expressing a polynucleotide encoding an influenza antigen of the invention after only a single round of enrichment. However, it can be more efficient to select a concentration such that multiple cells are present in each droplet and then screen droplets to identify those that contain expressing cells. In general, any appropriate screening procedure can be employed. For example, selection or detection of a detectable marker such as GFP can be used. Western blots or ELISA assays can be used. Individual droplets (100 μl) contain more than enough cells for performance of these assays. Multiple rounds of enrichment are performed to isolate successively higher expressing cell lines. Single clonal plant cell lines (i.e., populations derived from a single ancestral cell) can be generated by further limiting dilution using standard methods for single cell cloning. However, it is not necessary to isolate individual clonal lines. A population containing multiple clonal cell lines can be used for expression of a polynucleotide encoding one or more influenza antigen(s) of the invention.

In general, certain considerations described above for generation of clonal root lines apply to the generation of clonal plant cell lines. For example, a diversity of viral vectors containing one or more polynucleotide(s) encoding an influenza antigen(s) of the invention can be used as can combinations of multiple different vectors. Similar screening methods can be used. As in the case of clonal root lines and clonal root cell lines, cells of a clonal plant cell line are derived from a single ancestral cell that contains viral vector and may, therefore, contain viral vector since it will be replicated and will be transmitted during cell division. Thus a high proportion(e.g., at least 50%, at least 75%, at least 80%, at least 90%, at least 95%), all (100%), or substantially all (at least 98%) of cells will contain viral vector. It is noted that since viral vector is inherited by daughter cells within a clonal plant cell line, movement of viral vector among cells is not necessary to maintain viral vector. A clonal plant cell line can be used for production of a polypeptide encoding an influenza antigen of the invention as described below.

Clonal Plants

Clonal plants can be generated from clonal roots, clonal root cell lines, and/or clonal plant cell lines produced according to the various methods described above. Methods for the generation of plants from roots, root cell lines, and plant cell lines such as clonal root lines, clonal root cell lines, and clonal plant cell lines described herein are well known in the art (see, e.g., Peres et al., 2001, Plant Cell, Tissue, and Organ Culture, 65:37; and standard reference works on plant molecular biology and biotechnology cited elsewhere herein). The invention therefore provides a method of generating a clonal plant comprising steps of (i) generating a clonal root line, clonal root cell line, or clonal plant cell line according to any of the inventive methods described above; and (ii) generating a whole plant from a clonal root line, clonal root cell line, or clonal plant. The clonal plants may be propagated and grown according to standard methods.

As in the case of clonal root lines, clonal root cell lines, and clonal plant cell lines, cells of a clonal plant are derived from a single ancestral cell that contains viral vector and may, therefore, contain viral vector since it will be replicated and will be transmitted during cell division. Thus a high proportion(e.g., at least 50%, at least 75%, at least 80%, at least 90%, at least 95%), all (100%), or substantially all (at least 98%) of cells will contain viral vector. It is noted that since viral vector is inherited by daughter cells within the clonal plant, movement of viral vector is not necessary to maintain viral vector.

Sprouts and Sprouted Seedling Plant Expression Systems

Systems and reagents for generating a variety of sprouts and sprouted seedlings which are useful for production of influenza antigen(s) according to the present invention have been described previously and are known in the art (see, for example, PCT Publication WO 04/43886, which is incorporated herein by reference). The present invention further provides sprouted seedlings, which may be edible, as a biomass containing an influenza antigen. In certain aspects, biomass is provided directly for consumption of antigen containing compositions. In some aspects, biomass is processed prior to consumption, for example, by homogenizing, crushing, drying, or extracting. In certain aspects, influenza antigen is purified from biomass and formulated into a pharmaceutical composition.

Additionally provided are methods for producing influenza antigen(s) in sprouted seedlings that can be consumed or harvested live (e.g., sprouts, sprouted seedlings of the Brassica genus). In certain aspects, the present invention involves growing a seed to an edible sprouted seedling in a contained, regulatable environment (e.g., indoors, in a container, etc.). A seed can be a genetically engineered seed that contains an expression cassette encoding an influenza antigen, which expression is driven by an exogenously inducible promoter. A variety of exogenously inducible promoters can be used that are inducible, for example, by light, heat, phytohormones, nutrients, etc.

In related embodiments, the present invention provides methods of producing influenza antigen(s) in sprouted seedlings by first generating a seed stock for a sprouted seedling by transforming plants with an expression cassette that encodes influenza antigen using an Agrobacterium transformation system, wherein expression of an influenza antigen is driven by an inducible promoter. Transgenic seeds can be obtained from a transformed plant, grown in a contained, regulatable environment, and induced to express an influenza antigen.

In some embodiments, methods are provided that involves infecting sprouted seedlings with a viral expression cassette encoding an influenza antigen, expression of which may be driven by any of a viral promoter or an inducible promoter. Sprouted seedlings are grown for two to fourteen days in a contained, regulatable environment or at least until sufficient levels of influenza antigen have been obtained for consumption or harvesting.

The present invention further provides systems for producing influenza antigen(s) in sprouted seedlings that include a housing unit with climate control and a sprouted seedling containing an expression cassette that encodes one or more influenza antigens, wherein expression is driven by a constitutive or inducible promoter. The systems can provide unique advantages over the outdoor environment or greenhouse, which cannot be controlled. Thus, the present invention enables a grower to precisely time the induction of expression of influenza antigen. It can greatly reduce time and cost of producing influenza antigen(s).

In certain aspects, transiently transfected sprouts contain viral vector sequences encoding an inventive influenza antigen. Seedlings are grown for a time period so as to allow for production of viral nucleic acid in sprouts, followed by a period of growth wherein multiple copies of virus are produced, thereby resulting in production of influenza antigen(s).

In certain aspects, genetically engineered seeds or embryos that contain a nucleic acid encoding influenza antigen(s) are grown to sprouted seedling stage in a contained, regulatable environment. The contained, regulatable environment may be a housing unit or room in which seeds can be grown indoors. All environmental factors of a contained, regulatable environment may be controlled. Since sprouts do not require light to grow, and lighting can be expensive, genetically engineered seeds or embryos may be grown to sprouted seedling stage indoors in the absence of light.

Other environmental factors that can be regulated in a contained, regulatable environment of the present invention include temperature, humidity, water, nutrients, gas (e.g., O₂ or CO₂ content or air circulation), chemicals (small molecules such as sugars and sugar derivatives or hormones such as such as phytohormones gibberellic or absisic acid, etc.) and the like.

According to certain methods of the present invention, expression of a nucleic acid encoding an influenza antigen may be controlled by an exogenously inducible promoter. Exogenously inducible promoters are caused to increase or decrease expression of a nucleic acid in response to an external, rather than an internal stimulus. A number of environmental factors can act as inducers for expression of nucleic acids carried by expression cassettes of genetically engineered sprouts. A promoter may be a heat-inducible promoter, such as a heat-shock promoter. For example, using as heat-shock promoter, temperature of a contained environment may simply be raised to induce expression of a nucleic acid. Other promoters include light inducible promoters. Light-inducible promoters can be maintained as constitutive promoters if light in a contained regulatable environment is always on. Alternatively or additionally, expression of a nucleic acid can be turned on at a particular time during development by simply turning on the light. A promoter may be a chemically inducible promoter is used to induce expression of a nucleic acid. According to these embodiments, a chemical could simply be misted or sprayed onto seed, embryo, or seedling to induce expression of nucleic acid. Spraying and misting can be precisely controlled and directed onto target seed, embryo, or seedling to which it is intended. The contained environment is devoid of wind or air currents, which could disperse chemical away from intended target, so that the chemical stays on the target for which it was intended.

According to the present invention, time of expression is induced can be selected to maximize expression of an influenza antigen in sprouted seedling by the time of harvest. Inducing expression in an embryo at a particular stage of growth, for example, inducing expression in an embryo at a particular number of days after germination, may result in maximum synthesis of an influenza antigen at the time of harvest. For example, inducing expression from the promoter 4 days after germination may result in more protein synthesis than inducing expression from the promoter after 3 days or after 5 days. Those skilled in the art will appreciate that maximizing expression can be achieved by routine experimentation. In some methods, sprouted seedlings are harvested at about 1, 2, 3, 4, 5, 6, 7, 8, 9, 10, 11, or 12 days after germination.

In cases where the expression vector has a constitutive promoter instead of an inducible promoter, sprouted seedling may be harvested at a certain time after transformation of sprouted seedling. For example, if a sprouted seedling were virally transformed at an early stage of development, for example, at embryo stage, sprouted seedlings may be harvested at a time when expression is at its maximum post-transformation, e.g., at about 1, 2, 3, 4, 5, 6, 7, 8, 9, 10, 11, 12, 13, or 14 days post-transformation. It could be that sprouts develop one, two, three or more months post-transformation, depending on germination of seed.

Generally, once expression of influenza antigen(s) begins, seeds, embryos, or sprouted seedlings are allowed to grow until sufficient levels of influenza antigen(s) are expressed. In certain aspects, sufficient levels are levels that would provide a therapeutic benefit to a patient if harvested biomass were eaten raw. Alternatively or additionally, sufficient levels are levels from which influenza antigen can be concentrated or purified from biomass and formulated into a pharmaceutical composition that provides a therapeutic benefit to a patient upon administration. Typically, influenza antigen is not a protein expressed in sprouted seedling in nature. At any rate, influenza antigen is typically expressed at concentrations above that which would be present in a sprouted seedling in nature.

Once expression of influenza antigen is induced, growth is allowed to continue until sprouted seedling stage, at which time sprouted seedlings are harvested. Sprouted seedlings can be harvested live. Harvesting live sprouted seedlings has several advantages including minimal effort and breakage. Sprouted seedlings of the present invention may be grown hydroponically, making harvesting a simple matter of lifting the sprouted seedling from its hydroponic solution. No soil is required for growth of the sprouted seedlings of the invention, but may be provided if deemed necessary or desirable by the skilled artisan. Because sprouts can be grown without soil, no cleansing of sprouted seedling material is required at the time of harvest. Being able to harvest the sprouted seedling directly from its hydroponic environment without washing or scrubbing minimizes breakage of the harvested material. Breakage and wilting of plants induces apoptosis. During apoptosis, certain proteolytic enzymes become active, which can degrade pharmaceutical protein expressed in the sprouted seedling, resulting in decreased therapeutic activity of the protein. Apoptosis-induced proteolysis can significantly decrease yield of protein from mature plants. Using methods of the present invention, apoptosis may be avoided when no harvesting takes place until the moment proteins are extracted from the plant.

For example, live sprouts may be ground, crushed, or blended to produce a slurry of sprouted seedling biomass, in a buffer containing protease inhibitors. Buffer may be maintained at about 4° C. In some aspects, sprouted seedling biomass is air-dried, spray dried, frozen, or freeze-dried. As in mature plants, some of these methods, such as air-drying, may result in a loss of activity of pharmaceutical protein. However, because sprouted seedlings are very small and have a large surface area to volume ratio, this is much less likely to occur. Those skilled in the art will appreciate that many techniques for harvesting biomass that minimize proteolysis of expressed protein are available and could be applied to the present invention.

In some embodiments, sprouted seedlings are edible. In certain embodiments, sprouted seedlings expressing sufficient levels of influenza antigens are consumed upon harvesting (e.g., immediately after harvest, within minimal period following harvest) so that absolutely no processing occurs before sprouted seedlings are consumed. In this way, any harvest-induced proteolytic breakdown of influenza antigen before administration of influenza antigen to a patient in need of treatment is minimized. For example, sprouted seedlings that are ready to be consumed can be delivered directly to a patient. Alternatively or additionally, genetically engineered seeds or embryos are delivered to a patient in need of treatment and grown to sprouted seedling stage by a patient. In one aspect, a supply of genetically engineered sprouted seedlings is provided to a patient, or to a doctor who will be treating patients, so that a continual stock of sprouted seedlings expressing certain desirable influenza antigens may be cultivated. This may be particularly valuable for populations in developing countries, where expensive pharmaceuticals are not affordable or deliverable. The ease with which sprouted seedlings of the invention can be grown makes sprouted seedlings of the present invention particularly desirable for such developing populations.

The regulatable nature of the contained environment imparts advantages to the present invention over growing plants in the outdoor environment. In general, growing genetically engineered sprouted seedlings that express pharmaceutical proteins in plants provides a pharmaceutical product faster (because plants are harvested younger) and with less effort, risk, and regulatory considerations than growing genetically engineered plants. The contained, regulatable environment used in the present invention reduces or eliminates risk of cross-pollinating plants in nature.

For example, a heat inducible promoter likely would not be used outdoors because outdoor temperature cannot be controlled. The promoter would be turned on any time outdoor temperature rose above a certain level. Similarly, the promoter would be turned off every time outdoor temperature dropped. Such temperature shifts could occur in a single day, for example, turning expression on in the daytime and off at night. A heat inducible promoter, such as those described herein, would not even be practical for use in a greenhouse, which is susceptible to climatic shifts to almost the same degree as outdoors. Growth of genetically engineered plants in a greenhouse is quite costly. In contrast, in the present system, every variable can be controlled so that the maximum amount of expression can be achieved with every harvest.

In certain embodiments, sprouted seedlings of the present invention are grown in trays that can be watered, sprayed, or misted at any time during development of sprouted seedling. For example, a tray may be fitted with one or more watering, spraying, misting, and draining apparatus that can deliver and/or remove water, nutrients, chemicals etc. at specific time and at precise quantities during development of a sprouted seedling. For example, seeds require sufficient moisture to keep them damp. Excess moisture drains through holes in trays into drains in the floor of the room. Typically, drainage water is treated as appropriate for removal of harmful chemicals before discharge back into the environment.

Another advantage of trays is that they can be contained within a very small space. Since no light is required for sprouted seedlings to grow, trays containing seeds, embryos, or sprouted seedlings may be tightly stacked vertically on top of one another, providing a large quantity of biomass per unit floor space in a housing facility constructed specifically for these purposes. In addition, stacks of trays can be arranged in horizontal rows within the housing unit. Once seedlings have grown to a stage appropriate for harvest (about two to fourteen days) individual seedling trays are moved into a processing facility, either manually or by automatic means, such as a conveyor belt.

The system of the present invention is unique in that it provides a sprouted seedling biomass, which is a source of an influenza antigen(s). Whether consumed directly or processed into the form of a pharmaceutical composition, because sprouted seedlings are grown in a contained, regulatable environment, sprouted seedling biomass and/or pharmaceutical composition derived from biomass can be provided to a consumer at low cost. In addition, the fact that the conditions for growth of the sprouted seedlings can be controlled makes the quality and purity of product consistent. The contained, regulatable environment of the invention obviates many safety regulations of the EPA that can prevent scientists from growing genetically engineered agricultural products out of doors.

Transformed Sprouts

A variety of methods can be used to transform plant cells and produce genetically engineered sprouted seedlings. Two available methods for transformation of plants that require that transgenic plant cell lines be generated in vitro, followed by regeneration of cell lines into whole plants include Agrobacterium tumefaciens mediated gene transfer and microprojectile bombardment or electroporation. Viral transformation is a more rapid and less costly method of transforming embryos and sprouted seedlings that can be harvested without an experimental or generational lag prior to obtaining desired product. For any of these techniques, the skilled artisan would appreciate how to adjust and optimize transformation protocols that have traditionally been used for plants, seeds, embryos, or spouted seedlings.

Agrobacterium Transformation Expression Cassettes

Agrobacterium is a representative genus of the gram-negative family Rhizobiaceae. This species is responsible for plant tumors such as crown gall and hairy root disease. In dedifferentiated plant tissue, which is characteristic of tumors, amino acid derivatives known as opines are produced by the Agrobacterium and catabolized by the plant. The bacterial genes responsible for expression of opines are a convenient source of control elements for chimeric expression cassettes. According to the present invention, Agrobacterium transformation system may be used to generate edible sprouted seedlings, which are merely harvested earlier than mature plants. Agrobacterium transformation methods can easily be applied to regenerate sprouted seedlings expressing influenza antigens.

In general, transforming plants involves transformation of plant cells grown in tissue culture by co-cultivation with an Agrobacterium tumefaciens carrying a plant/bacterial vector. The vector contains a gene encoding an influenza antigen. The Agrobacterium transfers vector to plant host cell and is then eliminated using antibiotic treatment. Transformed plant cells expressing influenza antigen are selected, differentiated, and finally regenerated into complete plantlets (Hellens et al., 2000, Plant Molecular Biology, 42:819; Pilon-Smits et al., 1999, Plant Physiolog., 119:123; Barfield et al., 1991, Plant Cell Reports, 10:308; and Riva et al., 1998, J. Biotech., 1(3); each of which is incorporated by reference herein.

Expression vectors for use in the present invention include a gene (or expression cassette) encoding an influenza antigen designed for operation in plants, with companion sequences upstream and downstream of the expression cassette. The companion sequences are generally of plasmid or viral origin and provide necessary characteristics to the vector to transfer DNA from bacteria to the desired plant host.

The basic bacterial/plant vector construct may desirably provide a broad host range prokaryote replication origin, a prokaryote selectable marker. Suitable prokaryotic selectable markers include resistance toward antibiotics such as ampicillin or tetracycline. Other DNA sequences encoding additional functions that are well known in the art may be present in the vector.

Agrobacterium T-DNA sequences are required for Agrobacterium mediated transfer of DNA to the plant chromosome. The tumor-inducing genes of T-DNA are typically removed and replaced with sequences encoding an influenza antigen. T-DNA border sequences are retained because they initiate integration of the T-DNA region into the plant genome. If expression of influenza antigen is not readily amenable to detection, the bacterial/plant vector construct may include a selectable marker gene suitable for determining if a plant cell has been transformed, e.g., nptII kanamycin resistance gene. On the same or different bacterial/plant vector (Ti plasmid) are Ti sequences. Ti sequences include virulence genes, which encode a set of proteins responsible for excision, transfer and integration of T-DNA into the plant genome (Schell, 1987, Science, 237:1176). Other sequences suitable for permitting integration of heterologous sequence into the plant genome may include transposon sequences, and the like, for homologous recombination.

Certain constructs will include an expression cassette encoding an antigen protein. One, two, or more expression cassettes may be used in a given transformation. The recombinant expression cassette contains, in addition to an influenza antigen encoding sequence, at least the following elements: a promoter region, plant 5′ untranslated sequences, initiation codon (depending upon whether or not an expressed gene has its own), and transcription and translation termination sequences. In addition, transcription and translation terminators may be included in expression cassettes or chimeric genes of the present invention. Signal secretion sequences that allow processing and translocation of a protein, as appropriate, may be included in the expression cassette. A variety of promoters, signal sequences, and transcription and translation terminators are described (see, for example, Lawton et al., 1987, Plant Mol. Biol., 9:315; U.S. Pat. No. 5,888,789, incorporated herein by reference). In addition, structural genes for antibiotic resistance are commonly utilized as a selection factor (Fraley et al. 1983, Proc. Natl. Acad. Sci., USA, 80:4803, incorporated herein by reference). Unique restriction enzyme sites at the 5′ and 3′ ends of a cassette allow for easy insertion into a pre-existing vector. Other binary vector systems for Agrobacterium-mediated transformation, carrying at least one T-DNA border sequence are described in PCT/EP99/07414, incorporated herein by reference.

Regeneration

Seeds of transformed plants may be harvested, dried, cleaned, and tested for viability and for the presence and expression of a desired gene product. Once this has been determined, seed stock is typically stored under appropriate conditions of temperature, humidity, sanitation, and security to be used when necessary. Whole plants may then be regenerated from cultured protoplasts as described (see, e.g., Evans et al., Handbook of Plant Cell Cultures, Vol. 1: MacMillan Publishing Co. New York, 1983; and Vasil (ed.), Cell Culture and Somatic Cell Genetics of Plants, Acad. Press, Orlando, Fla., Vol. 1, 1984, and Vol. 111, 1986, incorporated herein by reference). In certain aspects, plants are regenerated only to sprouted seedling stage. In some aspects, whole plants are regenerated to produce seed stocks and sprouted seedlings are generated from seeds of the seed stock.

All plants from which protoplasts can be isolated and cultured to give whole, regenerated plants can be transformed by the present invention so that whole plants are recovered that contain a transferred gene. It is known that practically all plants can be regenerated from cultured cells or tissues, including, but not limited to, all major species of plants that produce edible sprouts. Some suitable plants include alfalfa, mung bean, radish, wheat, mustard, spinach, carrot, beet, onion, garlic, celery, rhubarb, a leafy plant such as cabbage or lettuce, watercress or cress, herbs such as parsley, mint, or clovers, cauliflower, broccoli, soybean, lentils, edible flowers such as sunflower etc.

Means for regeneration vary from one species of plants to the next. However, those skilled in the art will appreciate that generally a suspension of transformed protoplants containing copies of a heterologous gene is first provided. Callus tissue is formed and shoots may be induced from callus and subsequently rooted. Alternatively or additionally, embryo formation can be induced from a protoplast suspension. These embryos germinate as natural embryos to form plants. Steeping seed in water or spraying seed with water to increase the moisture content of the seed to between 35-45% initiates germination. For germination to proceed, seeds are typically maintained in air saturated with water under controlled temperature and airflow conditions. The culture media will generally contain various amino acids and hormones, such as auxin and cytokinins. It is advantageous to add glutamic acid and proline to the medium, especially for such species as alfalfa. Shoots and roots normally develop simultaneously. Efficient regeneration will depend on the medium, the genotype, and the history of the culture. If these three variables are controlled, then regeneration is fully reproducible and repeatable.

The mature plants, grown from transformed plant cells, are selfed and non-segregating, homozygous transgenic plants are identified. An inbred plant produces seeds containing inventive antigen-encoding sequences. Such seeds can be germinated and grown to sprouted seedling stage to produce influenza antigen(s) according to the present invention.

In related embodiments, seeds of the present invention may be formed into seed products and sold with instructions on how to grow seedlings to the appropriate sprouted seedling stage for administration or harvesting into a pharmaceutical composition. In some related embodiments, hybrids or novel varieties embodying desired traits may be developed from inbred plants of the invention.

Direct Integration

Direct integration of DNA fragments into the genome of plant cells by microprojectile bombardment or electroporation may be used in the present invention (see, e.g., Kikkert et al., 1999, In Vitro Cellular & Developmental Biology. Plant: Journal of the Tissue Culture Association. 35:43; Bates, 1994, Mol. Biotech., 2:135). More particularly, vectors that express influenza antigen(s) of the present invention can be introduced into plant cells by a variety of techniques. As described above, vectors may include selectable markers for use in plant cells. Vectors may include sequences that allow their selection and propagation in a secondary host, such as sequences containing an origin of replication and selectable marker. Typically, secondary hosts include bacteria and yeast. In one embodiment, a secondary host is bacteria (e.g., Escherichia coli, the origin of replication is a colE1-type origin of replication) and a selectable marker is a gene encoding ampicillin resistance. Such sequences are well known in the art and are commercially available (e.g., Clontech, Palo Alto, Calif. or Stratagene, La Jolla, Calif.).

Vectors of the present invention may be modified to intermediate plant transformation plasmids that contain a region of homology to an Agrobacterium tumefaciens vector, a T-DNA border region from Agrobacterium tumefaciens, and antigen encoding nucleic acids or expression cassettes described above. Further vectors may include a disarmed plant tumor inducing plasmid of Agrobacterium tumefaciens.

According to this embodiment, direct transformation of vectors invention may involve microinjecting vectors directly into plant cells by use of micropipettes to mechanically transfer recombinant DNA (see, e.g., Crossway, 1985, Mol. Gen. Genet., 202:179, incorporated herein by reference). Genetic material may be transferred into a plant cell using polyethylene glycols (see, e.g., Krens et al., 1982, Nature, 296:72). Another method of introducing nucleic acids into plants via high velocity ballistic penetration by small particles with a nucleic acid either within the matrix of small beads or particles, or on the surface (see, e.g., Klein et al., 1987, Nature, 327:70; and Knudsen et al., Planta, 185:330). Yet another method of introduction is fusion of protoplasts with other entities, either minicells, cells, lysosomes, or other fusible lipid-surfaced bodies (see, e.g., Fraley et al., 1982, Proc. Natl. Acad. Sci., USA, 79:1859). Vectors of the invention may be introduced into plant cells by electroporation (see, e.g., Fromm et al. 1985, Proc. Natl. Acad. Sci., USA, 82:5824). According to this technique, plant protoplasts are electroporated in the presence of plasmids containing a gene construct. Electrical impulses of high field strength reversibly permeabilize biomembranes allowing introduction of plasmids. Electroporated plant protoplasts reform the cell wall divide and form plant callus, which can be regenerated to form sprouted seedlings of the invention. Those skilled in the art will appreciate how to utilize these methods to transform plants cells that can be used to generate edible sprouted seedlings.

Viral Transformation

Similar to conventional expression systems, plant viral vectors can be used to produce full-length proteins, including full length antigen. According to the present invention, plant virus vectors may be used to infect and produce antigen(s) in seeds, embryos, sprouted seedlings, etc. Viral system that can be used to express everything from short peptides to large complex proteins. Specifically, using tobamoviral vectors is described (see, for example, McCormick et al., 1999, Proc. Natl. Acad. Sci., USA, 96:703; Kumagai et al. 2000, Gene, 245:169; and Verch et al., J. Immunol. Methods, 220:69; each of which is incorporated herein by reference). Thus, plant viral vectors have a demonstrated ability to express short peptides as well as large complex proteins.

In certain embodiments, transgenic sprouts, which express influenza antigen, are generated utilizing a host/virus system. Transgenic sprouts produced by viral infection provide a source of transgenic protein that has already been demonstrated to be safe. For example, sprouts are free of contamination with animal pathogens. Unlike, for example, tobacco, proteins from an edible sprout could at least in theory be used in oral applications without purification, thus significantly reducing costs. In addition, a virus/sprout system offers a much simpler, less expensive route for scale-up and manufacturing, since transgenes are introduced into virus, which can be grown up to a commercial scale within a few days. In contrast, transgenic plants can require up to 5-7 years before sufficient seeds or plant material is available for large-scale trials or commercialization.

According to the present invention, plant RNA viruses have certain advantages, which make them attractive as vectors for foreign protein expression. The molecular biology and pathology of a number of plant RNA viruses are well characterized and there is considerable knowledge of virus biology, genetics, and regulatory sequences. Most plant RNA viruses have small genomes and infectious cDNA clones are available to facilitate genetic manipulation. Once infectious virus material enters a susceptible host cell, it replicates to high levels and spreads rapidly throughout the entire sprouted seedling (one to ten days post inoculation). Virus particles are easily and economically recovered from infected sprouted seedling tissue. Viruses have a wide host range, enabling use of a single construct for infection of several susceptible species. These characteristics are readily transferable to sprouts.

Foreign sequences can be expressed from plant RNA viruses, typically by replacing one of viral genes with desired sequence, by inserting foreign sequences into the virus genome at an appropriate position, or by fusing foreign peptides to structural proteins of a virus. Moreover, any of these approaches can be combined to express foreign sequences by trans-complementation of vital functions of a virus. A number of different strategies exist as tools to express foreign sequences in virus-infected plants using tobacco mosaic virus (TMV), alfalfa mosaic virus (AlMV), and chimeras thereof.

The genome of AlMV is a representative of the Bromoviridae family of viruses and consists of three genomic RNAs (RNAs1-3) and subgenomic RNA (RNA4). Genomic RNAs1 and 2 encode virus replicase proteins P1 and 2, respectively. Genomic RNA3 encodes cell-to-cell movement protein P3 and coat protein (CP). CP is translated from subgenomic RNA4, which is synthesized from genomic RNA3, and is required to start infection. Studies have demonstrated the involvement of CP in multiple functions, including genome activation, replication, RNA stability, symptom formation, and RNA encapsidation (see e.g., Bol et al., 1971, Virology, 46:73; Van Der Vossen et al., 1994, Virology 202:891; Yusibov et al., Virology, 208:405; Yusibov et al., 1998, Virology, 242:1; Bol et al., (Review, 100 refs.), 1999, J. Gen. Virol., 80:1089; De Graaff, 1995, Virology, 208:583; Jaspars et al., 1974, Adv. Virus Res., 19:37; Loesch-Fries, 1985, Virology, 146:177; Neeleman et al., 1991, Virology, 181:687; Neeleman et al., 1993, Virology, 196: 883; Van Der Kuyl et al., 1991, Virology, 183:731; and Van Der Kuyl et al., 1991, Virology, 185:496).

Encapsidation of viral particles is typically required for long distance movement of virus from inoculated to un-inoculated parts of seed, embryo, or sprouted seedling and for systemic infection. According to the present invention, inoculation can occur at any stage of plant development. In embryos and sprouts, spread of inoculated virus should be very rapid. Virions of AlMV are encapsidated by a unique CP (24 kD), forming more than one type of particle. The size (30- to 60-nm in length and 18 nm in diameter) and shape (spherical, ellipsoidal, or bacilliform) of the particle depends on the size of the encapsidated RNA. Upon assembly, the N-terminus of the ALMV CP is thought to be located on the surface of the virus particles and does not appear to interfere with virus assembly (Bol et al., 1971, Virology, 6:73). Additionally, the ALMV CP with an additional 38-amino acid peptide at its N-terminus forms particles in vitro and retains biological activity (Yusibov et al., 1995, J. Gen. Virol., 77:567).

AlMV has a wide host range, which includes a number of agriculturally valuable crop plants, including plant seeds, embryos, and sprouts. Together, these characteristics make ALMV CP an excellent candidate as a carrier molecule and AlMV an attractive candidate vector for expression of foreign sequences in a plant at the sprout stage of development. Moreover, upon expression from a heterologous vector such as TMV, AlMV CP encapsidates TMV genome without interfering with virus infectivity (Yusibov et al., 1997, Proc. Natl. Acad. Sci., USA, 94:5784, incorporated herein by reference). This allows use of TMV as a carrier virus for AlMV CP fused to foreign sequences.

TMV, the prototype of tobamoviruses, has a genome consisting of a single plus-sense RNA encapsidated with a 17.0 kD CP, which results in rod-shaped particles (300 nm in length). CP is the only structural protein of TMV and is required for encapsidation and long distance movement of virus in an infected host (Saito et al., 1990, Virology, 176:329). 183 and 126 kD proteins are translated from genomic RNA and are required for virus replication (Ishikawa et al., 1986, Nucleic Acids Res., 14:8291). 30 kD protein is the cell-to-cell movement protein of virus (Meshi et al., 1987, EMBO J, 6:2557). Movement and coat proteins are translated from subgenomic mRNAs (Hunter et al., 1976, Nature, 260:759; Bruening et al., 1976, Virology, 71:498; and Beachy et al., 1976, Virology, 73:498; each of which is incorporated herein by reference).

Other methods of transforming plant tissues include transforming the flower of the plant. Transformation of Arabidopsis thaliana can be achieved by dipping plant flowers into a solution of Agrobacterium tumefaciens (Curtis et al., 2001, Transgenic Research, 10:363; Qing et al., 2000, Molecular Breeding: New Strategies in Plant Improvement, 1:67). Transformed plants are formed in the population of seeds generated by “dipped” plants. At a specific point during flower development, a pore exists in the ovary wall through which Agrobacterium tumefaciens gains access to the interior of the ovary. Once inside the ovary, the Agrobacterium tumefaciens proliferates and transforms individual ovules (Desfeux et al., 2000, Plant Physiology, 123:895). Transformed ovules follow the typical pathway of seed formation within the ovary.

### Production and Isolation of Antigen

In general, standard methods known in the art may be used for culturing or growing plants, plant cells, and/or plant tissues of the invention (e.g., clonal plants, clonal plant cells, clonal roots, clonal root lines, sprouts, sprouted seedlings, plants, etc.) for production of antigen(s). A wide variety of culture media and bioreactors have been employed to culture hairy root cells, root cell lines, and plant cells (see, for example, Giri et al., 2000, Biotechnol. Adv., 18:1; Rao et al., 2002, Biotechnol. Adv., 20:101; and references in both of the foregoing, all of which are incorporated herein by reference. Clonal plants may be grown in any suitable manner.

In a certain embodiments, influenza antigens of the invention may be produced by any known method. In some embodiments, an influenza antigen is expressed in a plant or portion thereof. Proteins are isolated and purified in accordance with conventional conditions and techniques known in the art. These include methods such as extraction, precipitation, chromatography, affinity chromatography, electrophoresis, and the like. The present invention involves purification and affordable scaling up of production of influenza antigen(s) using any of a variety of plant expression systems known in the art and provided herein, including viral plant expression systems described herein.

In many embodiments of the present invention, it will be desirable to isolate influenza antigen(s) for generation of antibody products and/or desirable to isolate influenza antibody or antigen binding fragment produced. Where a protein of the invention is produced from plant tissue(s) or a portion thereof, e.g., roots, root cells, plants, plant cells, that express them, methods described in further detail herein, or any applicable methods known in the art may be used for any of partial or complete isolation from plant material. Where it is desirable to isolate the expression product from some or all of plant cells or tissues that express it, any available purification techniques may be employed. Those of ordinary skill in the art are familiar with a wide range of fractionation and separation procedures (see, for example, Scopes et al., Protein Purification: Principles and Practice, 3^{rd} Ed., Janson et al., 1993; Protein Purification: Principles, High Resolution Methods, and Applications, Wiley-VCH, 1998; Springer-Verlag, NY, 1993; and Roe, Protein Purification Techniques, Oxford University Press, 2001; each of which is incorporated herein by reference). Often, it will be desirable to render the product more than about 50%, 60%, 70%, 80%, 85%, 90%, 91%, 92%, 93%, 94%, 95%, 96%, 97%, 98%, or 99% pure. See, e.g., U.S. Pat. Nos. 6,740,740 and 6,841,659 for discussion of certain methods useful for purifying substances from plant tissues or fluids.

Those skilled in the art will appreciate that a method of obtaining desired influenza antigen(s) product(s) is by extraction. Plant material (e.g., roots, leaves, etc.) may be extracted to remove desired products from residual biomass, thereby increasing the concentration and purity of product. Plants may be extracted in a buffered solution. For example, plant material may be transferred into an amount of ice-cold water at a ratio of one to one by weight that has been buffered with, e.g., phosphate buffer. Protease inhibitors can be added as required. Plant material can be disrupted by vigorous blending or grinding while suspended in buffer solution and extracted biomass removed by filtration or centrifugation. The product carried in solution can be further purified by additional steps or converted to a dry powder by freeze-drying or precipitation. Extraction can be carried out by pressing. Plants or roots can be extracted by pressing in a press or by being crushed as they are passed through closely spaced rollers. Fluids expressed from crushed plants or roots are collected and processed according to methods well known in the art. Extraction by pressing allows release of products in a more concentrated form. However, overall yield of product may be lower than if product were extracted in solution.

### Antibodies

The present invention provides pharmaceutical antigen and antibody proteins for therapeutic use, such as influenza antigen(s) (e.g., influenza protein(s) or an immunogenic portion(s) thereof, or fusion proteins comprising influenza antibody protein(s) or an antigen binding portion(s) thereof) active as antibody for therapeutic and/or prophylactic treatment of influenza infection. Further, the invention provides veterinary uses, as such influenza antigen is active in veterinary applications. In certain embodiments, influenza antigen(s) and/or antibodies may be produced by plant(s) or portion thereof (e.g., root, cell, sprout, cell line, plant, etc.) of the invention. In certain embodiments, provided influenza antigens and/or antibodies are expressed in plants, plant cells, and/or plant tissues (e.g., sprouts, sprouted seedlings, roots, root culture, clonal cells, clonal cell lines, clonal plants, etc.), and can be used directly from plant or partially purified or purified in preparation for pharmaceutical administration to a subject.

Monoclonal Antibodies

Various methods for generating monoclonal antibodies (MAbs) are now very well known in the art. The most standard monoclonal antibody generation techniques generally begin along the same lines as those for preparing polyclonal antibodies (Antibodies: A Laboratory Manual, Cold Spring Harbor Laboratory, 1988, which is hereby incorporated by reference). A polyclonal antibody response is initiated by immunizing an animal with an immunogenic anionic phospholipid and/or aminophospholipid composition and, when a desired titer level is obtained, the immunized animal can be used to generate MAbs. Typically, the particular screening and selection techniques disclosed herein are used to select antibodies with the sought after properties.

MAbs may be readily prepared through use of well-known techniques, such as those exemplified in U.S. Pat. Nos. 4,196,265, incorporated herein by reference. Typically, the technique involves immunizing a suitable animal with a selected immunogen composition to stimulate antibody producing cells. Rodents such as mice and rats are exemplary animals, however, the use of rabbit, sheep and frog cells is possible. The use of rats may provide certain advantages (Goding, 1986, pp. 60-61; incorporated herein by reference), but mice are sometimes preferred, with the BALB/c mouse often being most preferred as this is most routinely used and generally gives a higher percentage of stable fusions.

Following immunization, somatic cells with the potential for producing the desired antibodies, specifically B lymphocytes (B cells), are selected for use in the MAb generation and fusion with cells of an immortal myeloma cell, generally one of the same species as the animal that was immunized. Myeloma cell lines suited for use in hybridoma-producing fusion procedures typically are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984; each incorporated herein by reference). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F, 4B210 or one of the above listed mouse cell lines; and U-266, GM1500-GRG2, LICR-LON-HMy2 and UC729-6, are all useful in connection with human cell fusions.

This culturing provides a population of hybridomas from which specific hybridomas are selected, followed by serial dilution and cloning into individual antibody producing lines, which can be propagated indefinitely for production of antibody.

MAbs produced are generally be further purified, e.g., using filtration, centrifugation and various chromatographic methods, such as HPLC or affinity chromatography, all of which purification techniques are well known to those of skill in the art. These purification techniques each involve fractionation to separate the desired antibody from other components of a mixture. Analytical methods particularly suited to the preparation of antibodies include, for example, protein A-Sepharose and/or protein G-Sepharose chromatography.

Antibody Fragments and Derivatives

Irrespective of the source of the original antibody against a neuraminidase, either the intact antibody, antibody multimers, or any one of a variety of functional, antigen-binding regions of the antibody may be used in the present invention. Exemplary functional regions include scFv, Fv, Fab′, Fab and F(ab′).sub.2 fragments of antibodies. Techniques for preparing such constructs are well known to those in the art and are further exemplified herein.

The choice of antibody construct may be influenced by various factors. For example, prolonged half-life can result from the active readsorption of intact antibodies within the kidney, a property of the Fc piece of immunoglobulin. IgG based antibodies, therefore, are expected to exhibit slower blood clearance than their Fab′ counterparts. However, Fab′ fragment-based compositions will generally exhibit better tissue penetrating capability.

Antibody fragments can be obtained by proteolysis of the whole immunoglobulin by the non-specific thiolprotease, papain. Papain digestion yields two identical antigen-binding fragments, termed “Fab fragments,” each with a single antigen-binding site, and a residual “Fc fragment.” The various fractions are separated by protein A-Sepharose or ion exchange chromatography.

The usual procedure for preparation of F(ab′).sub.2 fragments from IgG of rabbit and human origin is limited proteolysis by the enzyme pepsin. Pepsin treatment of intact antibodies yields an F(ab′).sub.2 fragment that has two antigen-combining sites and is still capable of cross-linking antigen.

A Fab fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. F(ab′).sub.2 antibody fragments were originally produced as pairs of Fab′ fragments that have hinge cysteines between them. Other chemical couplings of antibody fragments are known.

An “Fv” fragment is the minimum antibody fragment that contains a complete antigen-recognition and binding site. This region consists of a dimer of one heavy chain and one light chain variable domain in tight, con-covalent association. It is in this configuration that three hypervariable regions of each variable domain interact to define an antigen-binding site on the surface of the V_{H}-V_{L} dimer. Collectively, six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “scFv” antibody fragments (now known as “single chains”) comprise the V_{H} and V_{L} domains of an antibody, wherein these domains are present in a single polypeptide chain. Generally, the Fv polypeptide further comprises a polypeptide linker between V_{H} and V_{L} domains that enables sFv to form the desired structure for antigen binding.

The following patents are incorporated herein by reference for the purposes of even further supplementing the present teachings regarding the preparation and use of functional, antigen-binding regions of antibodies, including scFv, Fv, Fab′, Fab and F(ab′).sub.2 fragments of antibodies: U.S. Pat. Nos. 5,855,866; 5,877,289; 5,965,132; 6,093,399; 6,261,535; and 6,004,555. WO 98/45331 is also incorporated herein by reference for purposes including even further describing and teaching the preparation of variable, hypervariable and complementarity determining (CDR) regions of antibodies.

“Diabodies” are small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between two domains on the same chain, the domains are forced to pair with the complementary domains of another chain and create two antigen-binding sites. Diabodies are described in EP 404,097 and WO 93/11161, each specifically incorporated herein by reference. “Linear antibodies,” which can be bispecific or monospecific, comprise a pair of tandem Fd segments (V.sub.H-C.sub.H1-V.sub.H-C.sub.H1) that form a pair of antigen binding regions, as described (see, for example, Zapata et al., 1995, incorporated herein by reference).

In using a Fab′ or antigen binding fragment of an antibody, with the attendant benefits on tissue penetration, one may derive additional advantages from modifying the fragment to increase its half-life. A variety of techniques may be employed, such as manipulation or modification of the antibody molecule itself, and conjugation to inert carriers. Any conjugation for the sole purpose of increasing half-life, rather than to deliver an agent to a target, should be approached carefully in that Fab′ and other fragments are chosen to penetrate tissues. Nonetheless, conjugation to non-protein polymers, such PEG and the like, is contemplated.

Modifications other than conjugation are therefore based upon modifying the structure of the antibody fragment to render it more stable, and/or to reduce the rate of catabolism in the body. One mechanism for such modifications is the use of D-amino acids in place of L-amino acids. Those of ordinary skill in the art will understand that the introduction of such modifications needs to be followed by rigorous testing of the resultant molecule to ensure that it still retains the desired biological properties. Further stabilizing modifications include the use of the addition of stabilizing moieties to either N-terminal or C-terminal, or both, which is generally used to prolong half-life of biological molecules. By way of example only, one may wish to modify termini by acylation or amination.

Bispecific Antibodies

Bispecific antibodies in general may be employed, so long as one arm binds to an aminophospholipid or anionic phospholipid and the bispecific antibody is attached, at a site distinct from the antigen binding site, to a therapeutic agent.

In general, the preparation of bispecific antibodies is well known in the art. One method involves the separate preparation of antibodies having specificity for the aminophospholipid or anionic phospholipid, on the one hand, and a therapeutic agent on the other. Peptic F(ab′)₂ fragments are prepared from two chosen antibodies, followed by reduction of each to provide separate Fab′_{SH} fragments. SH groups on one of two partners to be coupled are then alkylated with a cross-linking reagent such as O-phenylenedimaleimide to provide free maleimide groups on one partner. This partner may then be conjugated to the other by means of a thioether linkage, to give the desired F(ab′)₂ heteroconjugate. Other techniques are known wherein cross-linking with SPDP or protein A is carried out, or a trispecific construct is prepared.

One method for producing bispecific antibodies is by the fusion of two hybridomas to form a quadroma. As used herein, the term “quadroma” is used to describe the productive fusion of two B cell hybridomas. Using now standard techniques, two antibody producing hybridomas are fused to give daughter cells, and those cells that have maintained the expression of both sets of clonotype immunoglobulin genes are then selected.

CDR Technologies

Antibodies are comprised of variable and constant regions. The term “variable,” as used herein in reference to antibodies, means that certain portions of the variable domains differ extensively in sequence among antibodies, and are used in the binding and specificity of each particular antibody to its particular antigen. However, the variability is concentrated in three segments termed “hypervariable regions,” both in the light chain and the heavy chain variable domains.

The more highly conserved portions of variable domains are called the framework region (FR). Variable domains of native heavy and light chains each comprise four FRs (FR1, FR2, FR3 and FR4, respectively), largely adopting a beta-sheet configuration, connected by three hypervariable regions, which form loops connecting, and in some cases, forming part of, the beta-sheet structure.

The hypervariable regions in each chain are held together in close proximity by the FRs and, with hypervariable regions from the other chain, contribute to the formation of the antigen-binding site of antibodies (Kabat et al., 1991, incorporated herein by reference). Constant domains are not involved directly in binding an antibody to an antigen, but exhibit various effector functions, such as participation of the antibody in antibody-dependent cellular toxicity.

The term “hypervariable region,” as used herein, refers to amino acid residues of an antibody that are responsible for antigen-binding. The hypervariable region comprises amino acid residues from a “complementarity determining region” or “CDR” (i.e. residues 24-34 (L1), 50-56 (L2) and 89-97 (L3) in the light chain variable domain and 31-35 (H1), 50-56 (H2) and 95-102 (H3) in the heavy chain variable domain; Kabat et al., 1991, incorporated herein by reference) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain). “Framework” or “FR” residues are those variable domain residues other than the hypervariable region residues as herein defined.

The DNA and deduced amino acid sequences of Vh and V kappa chains of the 2B9 antibody encompass CDR1-3 of variable regions of heavy and light chains of the antibody. In light of the sequence and other information provided herein, and the knowledge in the art, a range of 2B9-like and improved antibodies and antigen binding regions can now be prepared and are thus encompassed by the present invention. Sequences of the 2B9 anti-N1 monoclonal antibody light and heavy chain variable regions are presented in Appendix A.

In certain embodiments, the invention provides at least one CDR of the antibody produced by the hybridoma 2B9, to be deposited. In some embodiments, the invention provides a CDR, antibody, or antigen binding region thereof, which binds to at least a neuraminidase, and which comprises at least one CDR of the antibody produced by the hybridoma 2B9, to be deposited.

In one particular embodiment, the invention provides an antibody, or antigen binding region thereof, in which the framework regions of the 2B9 antibody have been changed from mouse to a human IgG, such as human IgG1 or other IgG subclass to reduce immunogenicity in humans. In some embodiments, sequences of the 2B9 antibody are examined for the presence of T-cell epitopes, as is known in the art. The underlying sequence can then be changed to remove T-cell epitopes, i.e., to “deimmunize” the antibody.

The availability of DNA and amino acid sequences of Vh and V kappa chains of the 2B9 antibody means that a range of antibodies can now be prepared using CDR technologies. In particular, random mutations are made in the CDRs and products screened to identify antibodies with higher affinities and/or higher specificities. Such mutagenesis and selection is routinely practiced in the antibody arts. It is particularly suitable for use in the present invention, given the advantageous screening techniques disclosed herein. A convenient way for generating such substitutional variants is affinity maturation using phage display.

CDR shuffling and implantation technologies can be used with antibodies of the present invention, specifically 2B9 antibodies. CDR shuffling inserts CDR sequences into a specific framework region (Jirholt et al., 1998, incorporated herein by reference). CDR implantation techniques permit random combination of CDR sequences into a single master framework (Soderlind et al., 1999, 2000, each incorporated herein by reference). Using such techniques, CDR sequences of the 2B9 antibody, for example, are mutagenized to create a plurality of different sequences, which are incorporated into a scaffold sequence and the resultant antibody variants screened for desired characteristics, e.g., higher affinity.

Antibodies from Phagemid Libraries

Recombinant technology now allows the preparation of antibodies having a desired specificity from recombinant genes encoding a range of antibodies (Van Dijk et al., 1989; incorporated herein by reference). Certain recombinant techniques involve isolation of antibody genes by immunological screening of combinatorial immunoglobulin phage expression libraries prepared from RNA isolated from spleen of an immunized animal (Morrison et al., 1986; Winter and Milstein, 1991; Barbas et al., 1992; each incorporated herein by reference). For such methods, combinatorial immunoglobulin phagemid libraries are prepared from RNA isolated from spleen of an immunized animal, and phagemids expressing appropriate antibodies are selected by panning using cells expressing antigen and control cells. Advantage of this approach over conventional hybridoma techniques include approximately 10⁴ times as many antibodies can be produced and screened in a single round, and that new specificities are generated by H and L chain combination, which further increases the percentage of appropriate antibodies generated.

One method for the generation of a large repertoire of diverse antibody molecules in bacteria utilizes the bacteriophage lambda as the vector (Huse et al., 1989; incorporated herein by reference). Production of antibodies using the lambda vector involves the cloning of heavy and light chain populations of DNA sequences into separate starting vectors. Vectors are subsequently combined randomly to form a single vector that directs co-expression of heavy and light chains to form antibody fragments. The general technique for filamentous phage display is described (U.S. Pat. No. 5,658,727, incorporated herein by reference). In a most general sense, the method provides a system for the simultaneous cloning and screening of pre-selected ligand-binding specificities from antibody gene repertoires using a single vector system. Screening of isolated members of the library for a pre-selected ligand-binding capacity allows the correlation of the binding capacity of an expressed antibody molecule with a convenient means to isolate a gene that encodes the member from the library. Additional methods for screening phagemid libraries are described (U.S. Pat. Nos. 5,580,717; 5,427,908; 5,403,484; and 5,223,409, each incorporated herein by reference).

One method for the generation and screening of large libraries of wholly or partially synthetic antibody combining sites, or paratopes, utilizes display vectors derived from filamentous phage such as M13, fl or fd (U.S. Pat. No. 5,698,426, incorporated herein by reference). Filamentous phage display vectors, referred to as “phagemids,” yield large libraries of monoclonal antibodies having diverse and novel immunospecificities. The technology uses a filamentous phage coat protein membrane anchor domain as a means for linking gene-product and gene during the assembly stage of filamentous phage replication, and has been used for the cloning and expression of antibodies from combinatorial libraries (Kang et al., 1991; Barbas et al., 1991; each incorporated herein by reference). The surface expression library is screened for specific Fab fragments that bind neuraminidase molecules by standard affinity isolation procedures. The selected Fab fragments can be characterized by sequencing the nucleic acids encoding the polypeptides after amplification of the phage population.

One method for producing diverse libraries of antibodies and screening for desirable binding specificities is described (U.S. Pat. Nos. 5,667,988 and 5,759,817, each incorporated herein by reference). The method involves the preparation of libraries of heterodimeric immunoglobulin molecules in the form of phagemid libraries using degenerate oligonucleotides and primer extension reactions to incorporate degeneracies into CDR regions of immunoglobulin variable heavy and light chain variable domains, and display of mutagenized polypeptides on the surface of the phagemid. Thereafter, the display protein is screened for the ability to bind to a preselected antigen. A further variation of this method for producing diverse libraries of antibodies and screening for desirable binding specificities is described U.S. Pat. No. 5,702,892, incorporated herein by reference). In this method, only heavy chain sequences are employed, heavy chain sequences are randomized at all nucleotide positions which encode either the CDRI or CDRIII hypervariable region, and the genetic variability in the CDRs is generated independent of any biological process.

Transgenic Mice Containing Human Antibody Libraries

Recombinant technology is available for the preparation of antibodies. In addition to the combinatorial immunoglobulin phage expression libraries disclosed above, one molecular cloning approach is to prepare antibodies from transgenic mice containing human antibody libraries. Such techniques are described (U.S. Pat. No. 5,545,807, incorporated herein by reference).

In a most general sense, these methods involve the production of a transgenic animal that has inserted into its germline genetic material that encodes for at least part of an immunoglobulin of human origin or that can rearrange to encode a repertoire of immunoglobulins. The inserted genetic material may be produced from a human source, or may be produced synthetically. The material may code for at least part of a known immunoglobulin or may be modified to code for at least part of an altered immunoglobulin.

The inserted genetic material is expressed in the transgenic animal, resulting in production of an immunoglobulin derived at least in part from the inserted human immunoglobulin genetic material. The inserted genetic material may be in the form of DNA cloned into prokaryotic vectors such as plasmids and/or cosmids. Larger DNA fragments are inserted using yeast artificial chromosome vectors (Burke et al., 1987; incorporated herein by reference), or by introduction of chromosome fragments (Richer et al., 1989; incorporated herein by reference). The inserted genetic material may be introduced to the host in conventional manner, for example by injection or other procedures into fertilized eggs or embryonic stem cells.

Once a suitable transgenic animal has been prepared, the animal is simply immunized with the desired immunogen. Depending on the nature of the inserted material, the animal may produce a chimeric immunoglobulin, e.g. of mixed mouse/human origin, where the genetic material of foreign origin encodes only part of the immunoglobulin; or the animal may produce an entirely foreign immunoglobulin, e.g. of wholly human origin, where the genetic material of foreign origin encodes an entire immunoglobulin.

Polyclonal antisera may be produced from the transgenic animal following immunization. Immunoglobulin-producing cells may be removed from the animal to produce the immunoglobulin of interest. Generally, monoclonal antibodies are produced from the transgenic animal, e.g., by fusing spleen cells from the animal with myeloma cells and screening the resulting hybridomas to select those producing the desired antibody. Suitable techniques for such processes are described herein.

In one approach, the genetic material may be incorporated in the animal in such a way that the desired antibody is produced in body fluids such as serum or external secretions of the animal, such as milk, colostrum or saliva. For example, by inserting in vitro genetic material encoding for at least part of a human immunoglobulin into a gene of a mammal coding for a milk protein and then introducing the gene to a fertilized egg of the mammal, e.g., by injection, the egg may develop into an adult female mammal producing milk containing immunoglobulin derived at least in part from the inserted human immunoglobulin genetic material. The desired antibody can then be harvested from the milk. Suitable techniques for carrying out such processes are known to those skilled in the art.

The foregoing transgenic animals are usually employed to produce human antibodies of a single isotype, more specifically an isotype that is essential for B cell maturation, such as IgM and possibly IgD. Another method for producing human antibodies is described in U.S. Pat. Nos. 5,545,806; 5,569,825; 5,625,126; 5,633,425; 5,661,016; and 5,770,429; each incorporated by reference, wherein transgenic animals are described that are capable of switching from an isotype needed for B cell development to other isotypes.

In the method described in U.S. Pat. Nos. 5,545,806; 5,569,825; 5,625,126; 5,633,425; 5,661,016; and 5,770,429, human immunoglobulin transgenes contained within a transgenic animal function correctly throughout the pathway of B-cell development, leading to isotype switching. Accordingly, in this method, these transgenes are constructed so as to produce isotype switching and one or more of the following: (1) high level and cell-type specific expression, (2) functional gene rearrangement, (3) activation of and response to allelic exclusion, (4) expression of a sufficient primary repertoire, (5) signal transduction, (6) somatic hypermutation, and (7) domination of the transgene antibody locus during the immune response.

Humanized Antibodies

Human antibodies generally have at least three potential advantages for use in human therapy. First, because the effector portion is human, it may interact better with other parts of the human immune system, e.g., to destroy target cells more efficiently by complement-dependent cytotoxicity (CDC) or antibody-dependent cellular cytotoxicity (ADCC). Second, the human immune system should not recognize the antibody as foreign. Third, half-life in human circulation will be similar to naturally occurring human antibodies, allowing smaller and less frequent doses to be given.

Various methods for preparing human antibodies are provided herein. In addition to human antibodies, “humanized” antibodies have many advantages. “Humanized” antibodies are generally chimeric or mutant monoclonal antibodies from mouse, rat, hamster, rabbit or other species, bearing human constant and/or variable region domains or specific changes. Techniques for generating a so-called “humanized” antibody are well known to those of skill in the art.

A number of methods have been described to produce humanized antibodies. Controlled rearrangement of antibody domains joined through protein disulfide bonds to form new, artificial protein molecules or “chimeric” antibodies can be utilized (Konieczny et al., 1981; incorporated herein by reference). Recombinant DNA technology can be used to construct gene fusions between DNA sequences encoding mouse antibody variable light and heavy chain domains and human antibody light and heavy chain constant domains (Morrison et al., 1984; incorporated herein by reference).

DNA sequences encoding antigen binding portions or complementarity determining regions (CDR's) of murine monoclonal antibodies can be grafted by molecular means into DNA sequences encoding frameworks of human antibody heavy and light chains (Jones et al., 1986; Riechmann et al., 1988; each incorporated herein by reference). Expressed recombinant products are called “reshaped” or humanized antibodies, and comprise the framework of a human antibody light or heavy chain and antigen recognition portions, CDR's, of a murine monoclonal antibody.

One method for producing humanized antibodies is described in U.S. Pat. No. 5,639,641, incorporated herein by reference. A similar method for the production of humanized antibodies is described in U.S. Pat. Nos. 5,693,762; 5,693,761; 5,585,089; and 5,530,101, each incorporated herein by reference. These methods involve producing humanized immunoglobulins having one or more complementarity determining regions (CDR's) and possible additional amino acids from a donor immunoglobulin and a framework region from an accepting human immunoglobulin. Each humanized immunoglobulin chain usually comprises, in addition to CDR's, amino acids from the donor immunoglobulin framework that are capable of interacting with CDR's to effect binding affinity, such as one or more amino acids that are immediately adjacent to a CDR in the donor immunoglobulin or those within about 3A as predicted by molecular modeling. Heavy and light chains may each be designed by using any one, any combination, or all of various position criteria described in U.S. Pat. Nos. 5,693,762; 5,693,761; 5,585,089; and 5,530,101, each incorporated herein by reference. When combined into an intact antibody, humanized immunoglobulins are substantially non-immunogenic in humans and retain substantially the same affinity as the donor immunoglobulin to the original antigen.

An additional method for producing humanized antibodies is described in U.S. Pat. Nos. 5,565,332 and 5,733,743, each incorporated herein by reference. This method combines the concept of humanizing antibodies with the phagemid libraries described herein. In a general sense, the method utilizes sequences from the antigen binding site of an antibody or population of antibodies directed against an antigen of interest. Thus for a single rodent antibody, sequences comprising part of the antigen binding site of the antibody may be combined with diverse repertoires of sequences of human antibodies that can, in combination, create a complete antigen binding site.

Antigen binding sites created by this process differ from those created by CDR grafting, in that only the portion of sequence of the original rodent antibody is likely to make contacts with antigen in a similar manner. Selected human sequences are likely to differ in sequence and make alternative contacts with the antigen from those of the original binding site. However, constraints imposed by binding of the portion of original sequence to antigen and shapes of the antigen and its antigen binding sites, are likely to drive new contacts of human sequences to the same region or epitope of the antigen. This process has therefore been termed “epitope imprinted selection,” or “EIS.”

Starting with an animal antibody, one process results in the selection of antibodies that are partly human antibodies. Such antibodies may be sufficiently similar in sequence to human antibodies to be used directly in therapy or after alteration of a few key residues. In EIS, repertoires of antibody fragments can be displayed on the surface of filamentous phase and genes encoding fragments with antigen binding activities selected by binding of the phage to antigen.

Yet additional methods for humanizing antibodies contemplated for use in the present invention are described in U.S. Pat. Nos. 5,750,078; 5,502,167; 5,705,154; 5,770,403; 5,698,417; 5,693,493; 5,558,864; 4,935,496; and 4,816,567, each incorporated herein by reference.

As discussed in the above techniques, the advent of methods of molecular biology and recombinant technology, it is now possible to produce antibodies for use in the present invention by recombinant means and thereby generate gene sequences that code for specific amino acid sequences found in the polypeptide structure of antibodies. This has permitted the ready production of antibodies having sequences characteristic of inhibitory antibodies from different species and sources, as discussed above. In accordance with the foregoing, the antibodies useful in the methods of the present invention are anti-neuraminidase antibodies, specifically antibodies whose specificity is toward the same epitope of neuraminidase as 2B9 and include all therapeutically active variants and antigen binding fragments thereof whether produced by recombinant methods or by direct synthesis of the antibody polypeptides.

The present invention provides plants, plant cells, and plant tissues expressing antibodies that maintain pharmaceutical activity when administered to a subject in need thereof. Exemplary subjects include vertebrates (e.g., mammals, such as humans). According to the present invention, subjects include veterinary subjects such as bovines, ovines, canines, felines, etc. In certain aspects, an edible plant or portion thereof (e.g., sprout, root) is administered orally to a subject in a therapeutically effective amount. In some aspects one or more influenza antibody is provided in a pharmaceutical preparation, as described herein.

### Therapeutic Compositions and Uses

According to the present invention, treatment of a subject with an influenza antibody is intended to elicit a physiological effect. A antibody or antigen binding fragment thereof may have healing curative or palliative properties against a disorder or disease and can be administered to ameliorate relieve, alleviate, delay onset of, reverse or lessen symptoms or severity of a disease or disorder. An antibody composition comprising an influenza antigen may have prophylactic properties and can be used to prevent or delay the onset of a disease or to lessen the severity of such disease, disorder, or pathological condition when it does emerge. A physiological effect elicited by treatment of a subject with antigen according to the present invention can include an effective immune response such that infection by an organism is thwarted.

In some embodiments, antibody compositions are delivered by oral and/or mucosal routes. Oral and/or mucosal delivery has the potential to prevent infection of mucosal tissues, the primary gateway of infection for many pathogens. Oral and/or mucosal delivery can prime systemic immune response. There has been considerable progress in the development of heterologous expression systems for oral administration of antigens that stimulate the mucosal-immune system and can prime systemic immunity. Previous efforts at delivery of oral protein however, have demonstrated a requirement for considerable quantities of antigen in achieving efficacy. Thus, economical production of large quantities of target antibody or antigen binding fragment(s) thereof is a prerequisite for creation of effective oral proteins. The development of plants expressing antibody, including thermostable antigens, represents a more realistic approach to such difficulties.

The pharmaceutical preparations of the present invention can be administered in a wide variety of ways to a subject, such as, for example, orally, nasally, enterally, parenterally, intramuscularly or intravenously, rectally, vaginally, topically, ocularly, pulmonarily, or by contact application. In certain embodiments, an influenza antigen expressed in a plant or portion thereof is administered to a subject orally by direct administration of a plant to a subject. In some aspects an antibody or antigen binding fragment thereof expressed in a plant or portion thereof is extracted and/or purified, and used for the preparation of a pharmaceutical composition. It may be desirable to formulate such isolated products for their intended use (e.g., as a pharmaceutical agent, antibody composition, etc.). In some embodiments, it will be desirable to formulate products together with some or all of plant tissues that express them.

Where it is desirable to formulate product together with the plant material, it will often be desirable to have utilized a plant that is not toxic to the relevant recipient (e.g., a human or other animal). Relevant plant tissue (e.g., cells, roots, leaves) may simply be harvested and processed according to techniques known in the art, with due consideration to maintaining activity of the expressed product. In certain embodiments of the invention, it is desirable to have expressed influenza antigen in an edible plant (and, specifically in edible portions of the plant) so that the material can subsequently be eaten. For instance, where antibody or antigen binding fragment thereof is active after oral delivery (when properly formulated), it may be desirable to produce antibody protein in an edible plant portion, and to formulate expressed influenza antibody for oral delivery together with some or all of the plant material with which the protein was expressed.

Antibody or antigen binding fragment thereof (i.e., influenza antibody or antigen binding fragment thereof of the invention) provided may be formulated according to known techniques. For example, an effective amount of an antibody product can be formulated together with one or more organic or inorganic, liquid or solid, pharmaceutically suitable carrier materials. An antibody or antigen binding fragment thereof produced according to the present invention may be employed in dosage forms such as tablets, capsules, troches, dispersions, suspensions, solutions, gelcaps, pills, caplets, creams, ointments, aerosols, powder packets, liquid solutions, solvents, diluents, surface active agents, isotonic agents, thickening or emulsifying agents, preservatives, and solid bindings, as long as the biological activity of the protein is not destroyed by such dosage form.

In general, compositions may comprise any of a variety of different pharmaceutically acceptable carrier(s), adjuvant(s), or vehicle(s), or a combination of one or more such carrier(s), adjuvant(s), or vehicle(s). As used herein the language “pharmaceutically acceptable carrier, adjuvant, or vehicle” includes solvents, dispersion media, coatings, antibacterial and antifungal agents, isotonic and absorption delaying agents, and the like, compatible with pharmaceutical administration. Materials that can serve as pharmaceutically acceptable carriers include, but are not limited to sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening agents, flavoring agents, and perfuming agents, preservatives, and antioxidants can be present in the composition, according to the judgment of the formulator (see also Remington's Pharmaceutical Sciences, Fifteenth Edition, E. W. Martin, Mack Publishing Co., Easton, Pa., 1975). For example, antibody or antigen binding fragment product may be provided as a pharmaceutical composition by means of conventional mixing granulating dragee-making, dissolving, lyophilizing, or similar processes.

Additional Components

Compositions may include additionally any suitable adjuvant to enhance the immunogenicity of the composition when administered to a subject. For example, such adjuvant(s) may include, without limitation, extracts of Quillaja saponaria (QS), including purified subfractions of food grade QS such as Quil A and QS-21, alum, aluminum hydroxide, aluminum phosphate, MF59, Malp2, incomplete Freund's adjuvant; Complete Freund's adjuvant; 3 De-O-acylated monophosphoryl lipid A (3D-MPL). Further adjuvants include immunomodulatory oligonucleotides, for example unmethylated CpG sequences as disclosed in WO 96/02555. Combinations of different adjuvants, such as those mentioned hereinabove, are contemplated as providing an adjuvant which is a preferential stimulator of TH1 cell response. For example, QS21 can be formulated together with 3D-MPL. The ratio of QS21:3D-MPL will typically be in the order of 1:10 to 10:1; 1:5 to 5:1; and often substantially 1:1. The preferred range for optimal synergy may be 2.5:1 to 1:1 3D-MPL: QS21. Doses of purified QS extracts suitable for use in a human formulation are from 0.01 mg to 10 mg per kilogram of body weight.

It should be noted that certain thermostable proteins (e.g., lichenase) may themselves demonstrate immunoresponse potentiating activity, such that use of such protein whether in a fusion with an influenza antigen or separately may be considered use of an adjuvant. Thus, compositions may further comprise one or more adjuvants. Certain compositions may comprise two or more adjuvants. Furthermore, depending on formulation and routes of administration, certain adjuvants may be preferred in particular formulations and/or combinations.

In certain situations, it may be desirable to prolong the effect of an antibody or antigen binding fragment thereof by slowing the absorption of one or more components of the antibody product (e.g., protein) that is subcutaneously or intramuscularly injected. This may be accomplished by use of a liquid suspension of crystalline or amorphous material with poor water solubility. The rate of absorption of product then depends upon its rate of dissolution, which in turn, may depend upon size and form. Alternatively or additionally, delayed absorption of a parenterally administered product is accomplished by dissolving or suspending the product in an oil vehicle. Injectable depot forms are made by forming microcapsule matrices of protein in biodegradable polymers such as polylactide-polyglycolide. Depending upon the ratio of product to polymer and the nature of the particular polymer employed, rate of release can be controlled. Examples of biodegradable polymers include poly(orthoesters) and poly(anhydrides). Depot injectable formulations may be prepared by entrapping product in liposomes or microemulsions, which are compatible with body tissues. Alternative polymeric delivery vehicles can be used for oral formulations. For example, biodegradable, biocompatible polymers such as ethylene vinyl acetate, polyanhydrides, polyglycolic acid, collagen, polyorthoesters, and polylactic acid, etc., can be used. Antigen(s) or an immunogenic portions thereof may be formulated as microparticles, e.g., in combination with a polymeric delivery vehicle.

Enterally administered preparations of antibody may be introduced in solid, semi-solid, suspension or emulsion form and may be compounded with any pharmaceutically acceptable carriers, such as water, suspending agents, and emulsifying agents. Antigens may be administered by means of pumps or sustained-release forms, especially when administered as a preventive measure, so as to prevent the development of disease in a subject or to ameliorate or delay an already established disease. Supplementary active compounds, e.g., compounds independently active against the disease or clinical condition to be treated, or compounds that enhance activity of an inventive compound, can be incorporated into or administered with compositions. Flavorants and coloring agents can be used.

Inventive antibody products, optionally together with plant tissue, are particularly well suited for oral administration as pharmaceutical compositions. Oral liquid formulations can be used and may be of particular utility for pediatric populations. Harvested plant material may be processed in any of a variety of ways (e.g., air drying, freeze drying, extraction etc.), depending on the properties of the desired therapeutic product and its desired form. Such compositions as described above may be ingested orally alone or ingested together with food or feed or a beverage. Compositions for oral administration include plants; extractions of plants, and proteins purified from infected plants provided as dry powders, foodstuffs, aqueous or non-aqueous solvents, suspensions, or emulsions. Examples of non-aqueous solvents are propylene glycol, polyethylene glycol, vegetable oil, fish oil, and injectable organic esters. Aqueous carriers include water, water-alcohol solutions, emulsions or suspensions, including saline and buffered medial parenteral vehicles including sodium chloride solution, Ringer's dextrose solution, dextrose plus sodium chloride solution, Ringer's solution containing lactose or fixed oils. Examples of dry powders include any plant biomass that has been dried, for example, freeze dried, air dried, or spray dried. For example, plants may be air dried by placing them in a commercial air dryer at about 120 degrees Fahrenheit until biomass contains less than 5% moisture by weight. The dried plants may be stored for further processing as bulk solids or further processed by grinding to a desired mesh sized powder. Alternatively or additionally, freeze-drying may be used for products that are sensitive to air-drying. Products may be freeze dried by placing them into a vacuum drier and dried frozen under a vacuum until the biomass contains less than about 5% moisture by weight. Dried material can be further processed as described herein.

Plant-derived material may be administered as or together with one or more herbal preparations. Useful herbal preparations include liquid and solid herbal preparations. Some examples of herbal preparations include tinctures, extracts (e.g., aqueous extracts, alcohol extracts), decoctions, dried preparations (e.g., air-dried, spray dried, frozen, or freeze-dried), powders (e.g., lyophilized powder), and liquid. Herbal preparations can be provided in any standard delivery vehicle, such as a capsule, tablet, suppository, liquid dosage, etc. Those skilled in the art will appreciate the various formulations and modalities of delivery of herbal preparations that may be applied to the present invention.

Inventive root lines, cell lines, plants, extractions, powders, dried preparations and purified protein or nucleic acid products, etc., can be in encapsulated form with or without one or more excipients as noted above. Solid dosage forms such as tablets, dragees, capsules, pills, and granules can be prepared with coatings and shells such as enteric coatings, release controlling coatings and other coatings well known in the pharmaceutical formulating art. In such solid dosage forms active agent may be mixed with at least one inert diluent such as sucrose, lactose or starch. Such dosage forms may comprise, as is normal practice, additional substances other than inert diluents, e.g., tableting lubricants and other tableting aids such as magnesium stearate and microcrystalline cellulose. In the case of capsules, tablets and pills, the dosage forms may comprise buffering agents. They may optionally contain opacifying agents and can be of a composition that they release the active ingredient(s) only, or preferentially, in a certain part of the intestinal tract, and/or in a delayed manner. Examples of embedding compositions that can be used include polymeric substances and waxes.

In some methods, a plant or portion thereof expressing an influenza antigen according to the present invention, or biomass thereof, is administered orally as medicinal food. Such edible compositions are typically consumed by eating raw, if in a solid form, or by drinking, if in liquid form. The plant material can be directly ingested without a prior processing step or after minimal culinary preparation. For example, the antibody protein may be expressed in a sprout which can be eaten directly. For instance, protein expressed in an alfalfa sprout, mung bean sprout, or spinach or lettuce leaf sprout, etc. In one embodiment, plant biomass may be processed and the material recovered after the processing step is ingested.

Processing methods useful in accordance with the present invention are methods commonly used in the food or feed industry. The final products of such methods typically include a substantial amount of an expressed antigen and can be conveniently eaten or drunk. The final product may be mixed with other food or feed forms, such as salts, carriers, favor enhancers, antibiotics, and the like, and consumed in solid, semi-solid, suspension, emulsion, or liquid form. Such methods can include a conservation step, such as, e.g., pasteurization, cooking, or addition of conservation and preservation agents. Any plant may be used and processed in the present invention to produce edible or drinkable plant matter. The amount of influenza antigen in a plant-derived preparation may be tested by methods standard in the art, e.g., gel electrophoresis, ELISA, or Western blot analysis, using a probe or antibody specific for product. This determination may be used to standardize the amount of antibody protein ingested. For example, the amount of antibody may be determined and regulated, for example, by mixing batches of product having different levels of product so that the quantity of material to be drunk or eaten to ingest a single dose can be standardized. The contained, regulatable environment of the present invention, however, should minimize the need to carry out such standardization procedures.

Antibody protein produced in a plant cell or tissue and eaten by a subject may be preferably absorbed by the digestive system. One advantage of the ingestion of plant tissue that has been only minimally processed is to provide encapsulation or sequestration of the protein in cells of the plant. Thus, product may receive at least some protection from digestion in the upper digestive tract before reaching the gut or intestine and a higher proportion of active product would be available for uptake.

Pharmaceutical compositions of the present invention can be administered therapeutically or prophylactically. The compositions may be used to treat or prevent a disease. For example, any individual who suffers from a disease or who is at risk of developing a disease may be treated. It will be appreciated that an individual can be considered at risk for developing a disease without having been diagnosed with any symptoms of the disease. For example, if the individual is known to have been, or to be intended to be, in situations with relatively high risk of exposure to influenza infection, that individual will be considered at risk for developing the disease. Similarly, if members of an individual's family or friends have been diagnosed with influenza infection, the individual may be considered to be at risk for developing the disease.

Liquid dosage forms for oral administration include, but are not limited to, pharmaceutically acceptable emulsions, microemulsions, solutions, suspensions, syrups, and elixirs. In addition to active agents, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents.

Compositions for rectal or vaginal administration may be suppositories or retention enemas, which can be prepared by mixing the compositions of this invention with suitable non-irritating excipients or carriers such as cocoa butter, polyethylene glycol or a suppository wax which are solid at ambient temperature but liquid at body temperature and therefore melt in the rectum or vaginal cavity and release the active protein.

Dosage forms for topical, transmucosal or transdermal administration of a composition of this invention include ointments, pastes, creams, lotions, gels, powders, solutions, sprays, inhalants or patches. The active agent, or preparation thereof, is admixed under sterile conditions with a pharmaceutically acceptable carrier and any needed preservatives or buffers as may be required. For transmucosal or transdermal administration, penetrants appropriate to the barrier to be permeated may be used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, antigen or an immunogenic portion thereof may be formulated into ointments, salves, gels, or creams as generally known in the art. Ophthalmic formulation, eardrops, and eye drops are contemplated as being within the scope of this invention. Additionally, the present invention contemplates the use of transdermal patches, which have the added advantage of providing controlled delivery of a protein to the body. Such dosage forms can be made by suspending or dispensing the product in the proper medium. Absorption enhancers can be used to increase the flux of the protein across the skin. The rate can be controlled by either providing a rate controlling membrane or by dispersing the protein in a polymer matrix or gel.

Inventive compositions are administered in such amounts and for such time as is necessary to achieve the desired result. In certain embodiments of the present invention a “therapeutically effective amount” of a pharmaceutical composition is that amount effective for treating, attenuating, or preventing a disease in a subject. Thus, the “amount effective to treat, attenuate, or prevent disease,” as used herein, refers to a nontoxic but sufficient amount of the pharmaceutical composition to treat, attenuate, or prevent disease in any subject. For example, the “therapeutically effective amount” can be an amount to treat, attenuate, or prevent infection (e.g., viral infection, influenza infection), etc.

The exact amount required may vary from subject to subject, depending on the species, age, and general condition of the subject, the stage of the disease, the particular pharmaceutical mixture, its mode of administration, and the like. Influenza antigens of the invention, including plants expressing antigen(s) and/or preparations thereof may be formulated in dosage unit form for ease of administration and uniformity of dosage. The expression “dosage unit form,” as used herein, refers to a physically discrete unit of composition appropriate for the patient to be treated. It will be understood, however, that the total daily usage of the compositions of the present invention is typically decided by an attending physician within the scope of sound medical judgment. The specific therapeutically effective dose level for any particular patient or organism may depend upon a variety of factors including the severity or risk of infection; the activity of the specific compound employed; the specific composition employed; the age, body weight, general health, sex of the patient, diet of the patient, pharmacokinetic condition of the patient, the time of administration, route of administration, and rate of excretion of the specific antigen(s) employed; the duration of the treatment; drugs used in combination or coincidental with the composition employed; and like factors well known in the medical arts.

It will be appreciated that compositions of the present invention can be employed in combination therapies (e.g., combination vaccine therapies), that is, pharmaceutical compositions can be administered concurrently with, prior to, or subsequent to, one or more other desired pharmaceutical and/or vaccination procedures. The particular combination of therapies (e.g., vaccines, therapeutic treatment of influenza infection) to employ in a combination regimen will generally take into account compatibility of the desired therapeutics and/or procedures and the desired therapeutic effect to be achieved. It will be appreciated that the therapies and/or vaccines employed may achieve a desired effect for the same disorder (for example, an inventive antigen may be administered concurrently with another influenza antibody), or they may achieve different effects.

### Kits

In one aspect, the present invention provides a pharmaceutical pack or kit including influenza antigens according to the present invention. In certain embodiments, pharmaceutical packs or kits include live sprouted seedlings, clonal entity or plant producing an antibody or antigen binding fragment according to the present invention, or preparations, extracts, or pharmaceutical compositions containing antibody in one or more containers filled with optionally one or more additional ingredients of pharmaceutical compositions of the invention. In some embodiments, pharmaceutical packs or kits include pharmaceutical compositions comprising purified influenza antigen according to the present invention, in one or more containers optionally filled with one or more additional ingredients of pharmaceutical compositions of the invention. In certain embodiments, the pharmaceutical pack or kit includes an additional approved therapeutic agent (e.g., influenza antibody, influenza vaccine) for use as a combination therapy. Optionally associated with such container(s) can be a notice in the form prescribed by a governmental agency regulating the manufacture, use or sale of pharmaceutical products, which notice reflects approval by the agency of manufacture, use, or sale for human administration.

Kits are provided that include therapeutic reagents. As but one non-limiting example, influenza antibody can be provided as oral formulations and administered as therapy. Alternatively or additionally, influenza antibody can be provided in an injectable formulation for administration. In one embodiment, influenza antibody can be provided in an inhalable formulation for administration. Pharmaceutical doses or instructions therefore may be provided in the kit for administration to an individual suffering from or at risk for influenza infection.

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The following examples contain information, exemplification and guidance, which can be adapted to the practice of this invention in its various embodiments and the equivalents thereof.

### Exemplification

### EXAMPLE 1

### Generation of Antigen Constructs

### Generation of Antigen Sequences from Influenza Virus Neuraminidase

Nucleotide sequence encoding neuraminidase of each of influenza virus type Vietnam H5N1 (NAV) and Wyoming H3N2 (NAW) was synthesized and confirmed as being correct. Produced nucleic acid was digested with restriction endonuclease SalI, sites for which had been engineered onto either end of sequence encoding domains. The resulting DNA fragments were fused in frame into the C-terminus to sequence encoding an engineered thermostable carrier molecule.

NAV(N1): (SEQ ID NO.: 27):
GGATCCTTAATTAAAATGGGATTCGTGCTTTTCTCTCAGCTTCCTTCTTTCCTTCTTGTGT
CTACTCTTCTTCTTTTCCTTGTGATTTCTCACTCTTGCCGTGCTCAAAATGTCGACCTTAT
GCTTCAGATTGGAAACATGATTTCTATTTGGGTGTCACACTCTATTCACACTGGAAACCA
GCATCAGTCTGAGCCAATTTCTAACACTAACCTTTTGACTGAGAAGGCTGTGGCTTCTGT
TAAGTTGGCTGGAAACTCTTCTCTTTGCCCTATTAACGGATGGGCTGTGTACTCTAAGGA
TAACTCTATTAGGATTGGATCTAAGGGAGATGTGTTCGTGATTAGGGAGCCATTCATTT
CTTGCTCTCACCTTGAGTGCCGTACTTTCTTCCTTACTCAGGGTGCTCTTCTTAACGATAA
GCACTCTAACGGAACTGTGAAGGATAGGTCTCCACACAGGACTCTTATGTCTTGTCCAG
TTGGAGAAGCTCCATCTCCATACAACTCTAGATTCGAGTCTGTTGCTTGGAGTGCTTCTG
CTTGCCATGATGGAACTTCATGGCTTACTATTGGAATTTCTGGACCAGATAACGGAGCT
GTTGCTGTGCTTAAGTACAACGGAATTATTACTGATACCATCAAGTCTTGGAGGAACAA
CATTCTTAGGACTCAGGAGTCTGAGTGTGCTTGCGTTAACGGATCTTGCTTCACTGTGAT
GACTGATGGACCATCTAATGGACAGGCTTCTCACAAGATTTTCAAGATGGAGAAGGGA
AAGGTTGTGAAGTCTGTGGAACTTGATGCTCCAAACTACCATTACGAGGAGTGTTCTTG
CTATCCAGATGCTGGAGAGATTACTTGTGTGTGCCGTGATAATTGGCATGGATCTAACA
GGCCATGGGTGTCATTCAATCAGAACCTTGAGTACCAGATTGGTTACATTTGCTCTGGA
GTGTTCGGAGATAATCCAAGGCCAAACGATGGAACTGGATCTTGTGGACCAGTGTCATC
TAATGGAGCTGGAGGAGTGAAGGGATTCTCTTTCAAGTACGGAAACGGAGTTTGGATTG
GAAGGACTAAGTCTACTAACTCTAGGAGTGGATTCGAGATGATTTGGGACCCAAACGG
ATGGACTGAGACTGATTCTTCTTTCTCTGTGAAGCAGGATATTGTGGCTATTACTGATTG
GAGTGGATACTCTGGATCTTTCGTTCAGCACCCAGAGCTTACTGGACTTGATTGCATTAG
GCCATGCTTCTGGGTTGAACTTATTAGGGGAAGGCCAAAGGAGTCTACTATTTGGACTT
CTGGATCTTCTATTTCTTTCTGCGGAGTGAATTCTGATACTGTGGGATGGTCTTGGCCAG
ATGGAGCTGAGCTTCCATTCACTATTGATAAGGTCGACCATCATCATCATCACCACAAG
GATGAGCTTTGACTCGAG
NAV: (SEQ ID NO.: 16):
LMLQIGNMISIWVSHSIHTGNQHQSEPISNTNLLTEKAVASVKLAGNSSLCPINGWAVYSKD
NSIRIGSKGDVFVIREPFISCSHLECRTFFLTQGALLNDKHSNGTVKDRSPHRTLMSCPVGEA
PSPYNSRFESVAWSASACHDGTSWLTIGISGPDNGAVAVLKYNGIITDTIKSWRNNILRTQES
ECACVNGSCFTVMTDGPSNGQASHKIFKMEKGKVVKSVELDAPNYHYEECSCYPDAGEIT
CVCRDNWHGSNRPWVSFNQNLEYQIGYICSGVFGDNPRPNDGTGSCGPVSSNGAGGVKGF
SFKYGNGVWIGRTKSTNSRSGFEMIWDPNGWTETDSSFSVKQDIVAITDWSGYSGSFVQHP
ELTGLDCIRPCFWVELIRGRPKESTIWTSGSSISFCGVNSDTVGWSWPDGAELPFTIDK
NAW(N2): (SEQ ID NO.: 28):
GGATCCTTAATTAAAATGGGATTCGTGCTTTTCTCTCAGCTTCCTTCTTTCCTTCTTGTGT
CTACTCTTCTTCTTTTCCTTGTGATTTCTCACTCTTGCCGTGCTCAAAATGTCGACAAGCA
GTACGAGTTCAACTCTCCACCAAACAACCAGGTTATGCTTTGCGAGCCAACTATTATTG
AGAGGAACATTACTGAGATTGTGTACCTTACTAACACTACTATTGAGAAGGAGATTTGC
CCAAAGTTGGCTGAGTACCGTAATTGGTCTAAGCCACAGTGCAACATTACTGGATTCGC
TCCATTCTCTAAGGATAACTCAATTAGGCTTTCTGCTGGAGGAGATATTTGGGTTACAA
GGGAGCCATACGTTTCTTGCGATCCAGATAAGTGCTACCAGTTCGCTCTTGGACAAGGA
ACTACTCTTAACAACGTGCACTCTAACGATACTGTGCACGATAGGACTCCATACCGTAC
TCTTTTGATGAACGAGCTTGGAGTTCCATTCCACCTTGGAACTAAGCAAGTGTGCATTGC
TTGGTCATCTTCATCTTGCCACGATGGAAAGGCTTGGCTTCATGTTTGCGTGACTGGAGA
TGATGAGAACGCTACTGCTTCTTTCATCTACAACGGAAGGCTTGTGGATTCTATTGTTTC
TTGGTCTAAGAAGATTCTTAGGACTCAGGAGTCTGAGTGTGTGTGCATTAACGGAACTT
GCACTGTGGTTATGACTGATGGATCTGCTTCTGGAAAGGCTGATACAAAGATTCTTTTC
ATTGAGGAGGGAAAGATTGTGCACACTTCTACTCTTTCTGGATCTGCTCAGCATGTTGA
GGAGTGTTCTTGCTACCCAAGGTATCCAGGAGTTAGATGTGTGTGCCGTGATAACTGGA
AGGGATCTAACAGGCCAATTGTGGATATTAACATTAAGGATTACTCTATTGTGTCATCTT
ATGTGTGCTCTGGACTTGTTGGAGATACTCCAAGGAAGAACGATTCTTCTTCATCTTCAC
ACTGCCTTGATCCAAATAACGAGGAGGGAGGACATGGAGTTAAGGGATGGGCTTTCGA
TGATGGAAACGATGTTTGGATGGGAAGGACTATTTCTGAGAAGTTGAGGAGCGGATAC
GAGACTTTCAAAGTGATTGAGGGATGGTCTAACCCAAATTCTAAGCTGCAGATTAACAG
GCAAGTGATTGTGGATAGGGGAAACAGGAGTGGATACTCTGGAATTTTCTCTGTGGAGG
GAAAGTCTTGCATTAACAGATGCTTCTACGTGGAGCTTATTAGGGGAAGGAAGCAGGA
GACTGAGGTTTTGTGGACTTCTAACTCTATTGTGGTGTTCTGCGGAACTTCTGGAACTTA
CGGAACTGGATCTTGGCCAGATGGAGCTGATATTAACCTTATGCCAATTGTCGACCATC
ATCACCATCACCACAAGGATGAGCTTTGACTCGAG
NAW: (SEQ ID NO.: 18):
KQYEFNSPPNNQVMLCEPTIIERNITEIVYLTNTTIEKEICPKLAEYRNWSKPQCNITGFAPFS
KDNSIRLSAGGDIWVTREPYVSCDPDKCYQFALGQGTTLNNVHSNDTVHDRTPYRTLLMN
ELGVPFHLGTKQVCIAWSSSSCHDGKAWLHVCVTGDDENATASFIYNGRLVDSIVSWSKKI
LRTQESECVCINGTCTVVMTDGSASGKADTKILFIEEGKIVHTSTLSGSAQHVEECSCYPRYP
GVRCVCRDNWKGSNRPIVDINIKDYSIVSSYVCSGLVGDTPRKNDSSSSSHCLDPNNEEGGH
GVKGWAFDDGNDVWMGRTISEKLRSGYETFKVIEGWSNPNSKLQINRQVIVDRGNRSGYS
GIFSVEGKSCINRCFYVELIRGRKQETEVLWTSNSIVVFCGTSGTYGTGSWPDGADINLMPI

### Generation of Recombinant Antigen Constructs

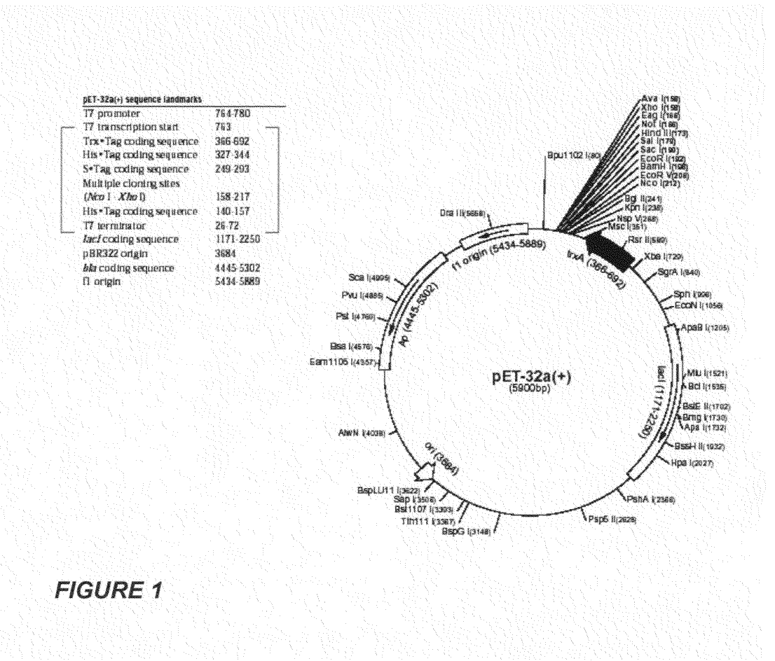

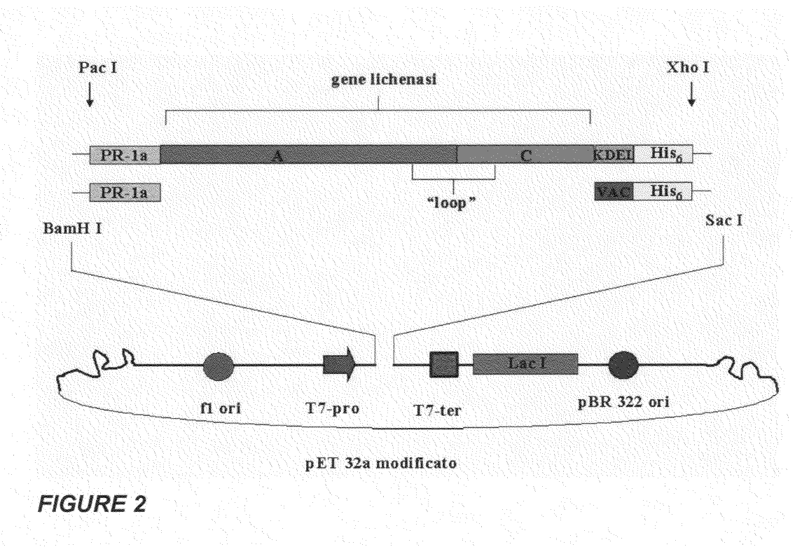

We used pET expression vectors, derived from pBR322 plasmid, engineered to take advantage of the features of the T7 bacteriophage gene 10 that promote high-level transcription and translation. The bacteriophage encoded RNA polymerase is highly specific for the T7 promoter sequences, which are rarely encountered in genomes other than T7 phage genome (FIG. 1). pET-32 has been used for fusing the HA and NA constructs into the loop region of a modified lichenase sequence that had been cloned in this vector. The catalytic domain of the lichenase gene with the upstream sequence PR-1A (“Pathogen-Related protein 1 A”), with a endoplasmic reticulum (KDEL) or a vacuolar retaining sequence (VAC) and a downstream His₆ tag were cloned between the PacI and XhoI sites in a modified pET-32 vector (in which the region between the T7 promoter and the T7 terminator had been excised). In this way the pET-PR-LicKM-KDEL and pET-PR-LicKM-VAC were obtained (FIG. 2). The DNA fragment HA domain or NA was subcloned into the 1 portion of LicKM to give a fusion in the correct reading frame for translation. Furthermore, LicKM-NA fusions were constructed. The DNA fragment of NAW or NAV was subcloned into the C-terminus of LicKM using a SalI site to give a fusion in the correct reading frame for translation.

### EXAMPLE 2

### Generation of Antigen Vectors

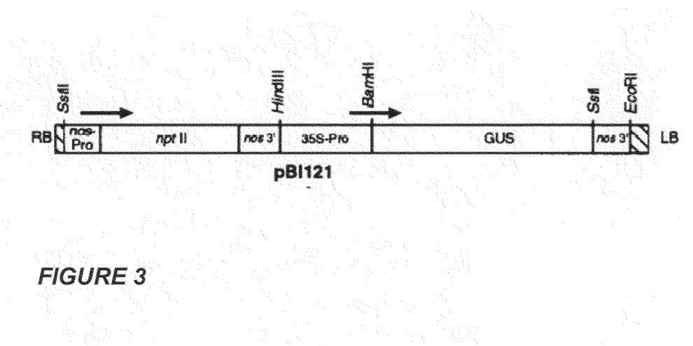

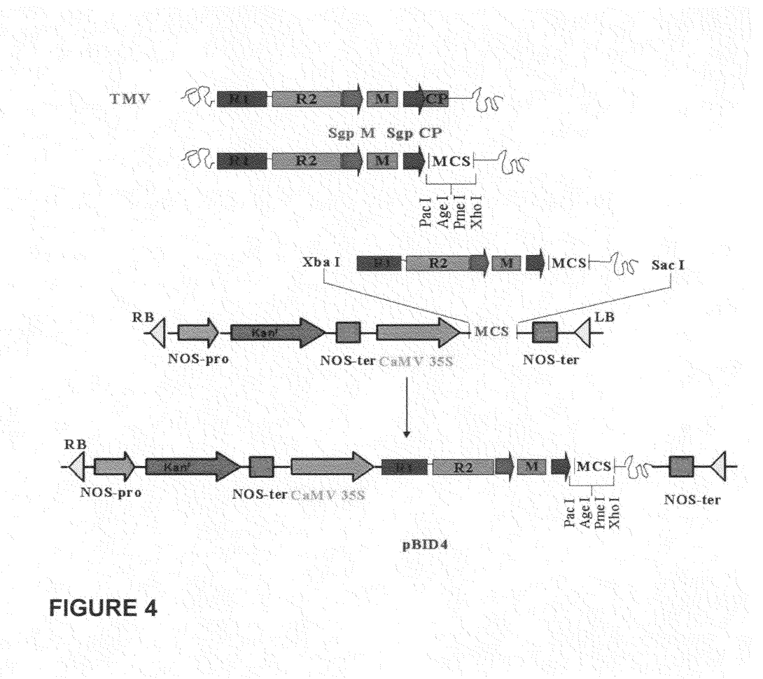

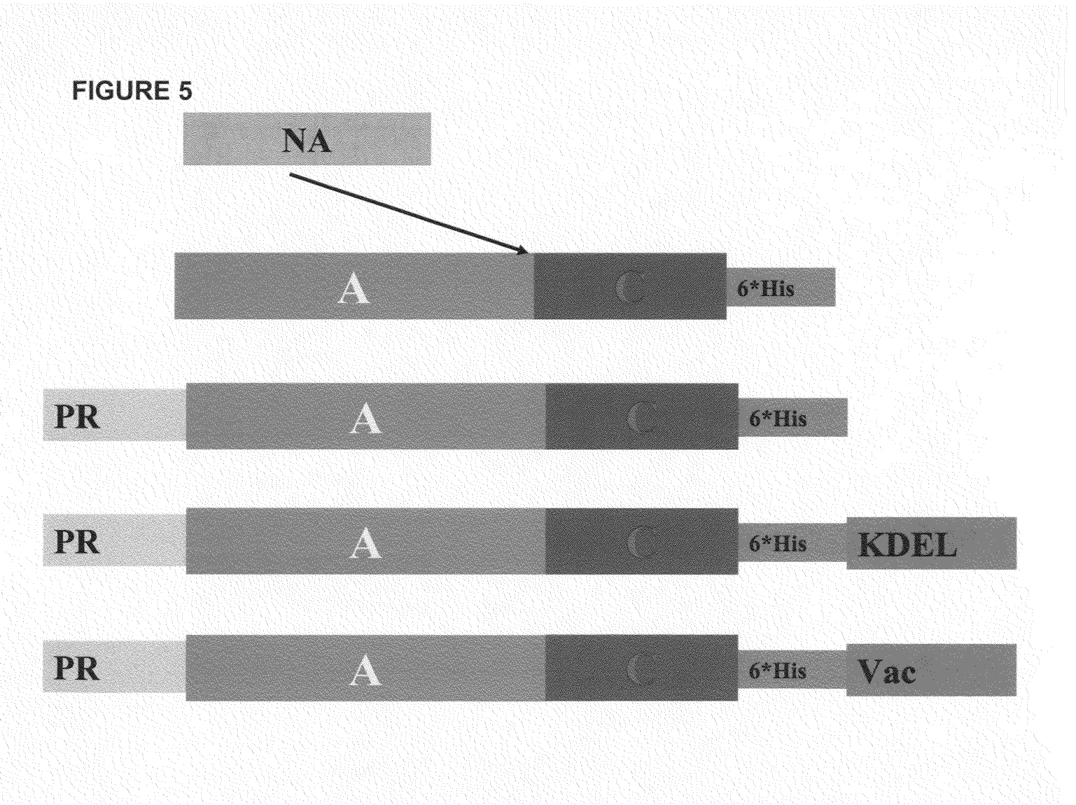

Target antigen constructs LicKM-NA was subcloned into the chosen viral vector (pBI-D4). pBI-D4 is a pBI121-derived binary vector in which the reporter gene coding for the Escherichia coli beta-D-glucuronidase (GUS) has been replaced by a “polylinker” where, between the XbaI and SacI sites, a TMV-derived vector has been cloned (FIG. 3). pBI-D4 is a TMV-based construct in which a foreign gene to be expressed (e.g., target antigen (e.g., LicKM-HA, LicKM-NA) replaces the coat protein (CP) gene of TMV. The virus retains the TMV 126/183kDa gene, the movement protein (MP) gene, and the CP subgenomic mRNA promoter (sgp), which extends into the CP open reading frame (ORF). The start codon for CP has been mutated. The virus lacks CP and therefore cannot move throughout the host plant via phloem. However, cell-to-cell movement of viral infection remains functional, and the virus can move slowly to the upper leaves in this manner. A multiple cloning site (PacI-PmeI-AgeI-XhoI) has been engineered at the end of sgp for expression of foreign genes, and is followed by the TMV 3′ non-translated region (NTR). The 35S promoter is fused at the 5′ end of the viral sequence. The vector sequence is positioned between the BamH1 and Sac1 sites of pBI121. The hammerhead ribozyme is placed 3′ of the viral sequence. (Chen et al., 2003, Mol. Breed., 11:287). These constructs include fusions of sequences encoding LicKM-HA or NA, to sequences encoding the signal peptide from tobacco PR-1a protein, a 6× His tag and the ER-retention anchor sequence KDEL or vacuolar sequence (FIG. 4). For constructs that contain sequence encoding, PR-LicKM-HA(SD)-KDEL, PR-LicKM-HA(GD)-KDEL, and PR-LicKM-NA-KDEL the coding DNA was introduced as PacI-XhoI fragments into pBI-D4. Furthermore, HAW (HA Wyoming), HAV (HA Vietnam), NAW (NA Wyoming), and NAV (NA Vietnam) were introduced directly as PacI-XhoI fragments into pBI-D4. Nucleotide sequence was subsequently verified spanning the subcloning junctions of the final expression constructs (FIG. 5).

### EXAMPLE 3

### Generation of Plants and Antigen Production

### Agrobacterium Infiltration of Plants

Agrobacterium-mediated transient expression system achieved by Agrobacterium infiltration can be utilized (Turpen et al., 1993, J. Virol. Methods, 42:227). Healthy leaves of N. benthamiana were infiltrated with A. rhizogenes containing viral vectors engineered to express LicKM-HA or LicKM-NA.

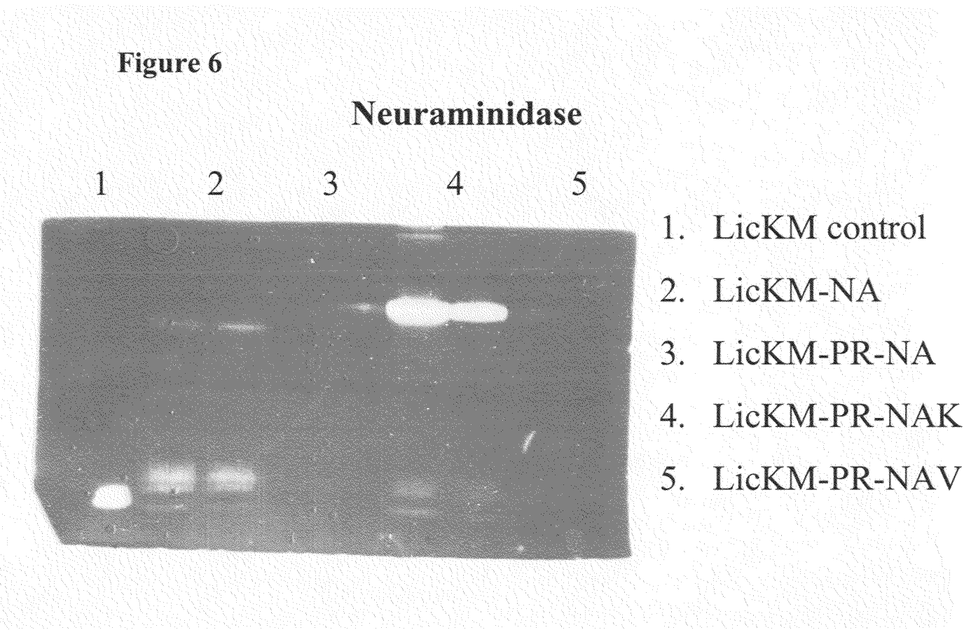

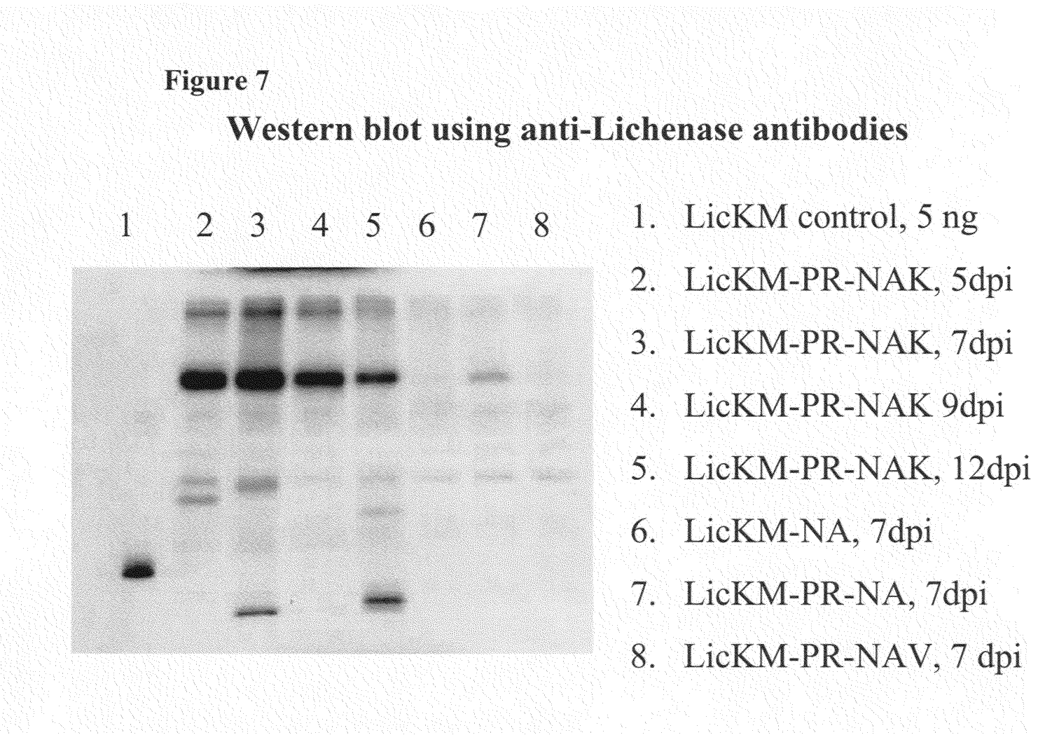

The A. rhizogenes strain A4 (ATCC 43057) was transformed with the constructs pBI-D4-PR-LicKM-HA-KDEL, pBI-D4-PR-LicKM-HA-VAC, pBI-D4-PR-LicKM-NA-KDEL and pBI-D4-PR-LicKM-NA-VAC. Agrobacterium cultures were grown and induced as described (Kapila et al. 1997, Plant Sci., 122:101). A 2 ml starter-culture (picked from a fresh colony) was grown overnight in YEB (5 g/l beef extract, 1 g/l yeast extract, 5 g/l peptone, 5 g/l sucrose, 2 mM MgSO₄) with 25 μg/ml kanamycin at 28° C. The starter culture was diluted 1:500 into 500 ml of YEB with 25 μg/ml kanamycin, 10 mM 2-4(-morpholino)ethanesulfonic acid (MES) pH 5.6, 2 mM additional MgSO₄ and 20 μM acetosyringone. The diluted culture was then grown overnight to an O.D.₆₀₀ of ˜1.7 at 28° C. The cells were centrifuged at 3,000×g for 15 minutes and re-suspended in MMA medium (MS salts, 10 mM MES pH 5.6, 20 g/l sucrose, 200 μM acetosyringone) to an O.D.₆₀₀ of 2.4, kept for 1-3 hour at room temperature, and used for Agrobacterium-infiltration. N. benthamiana leaves were injected with the Agrobacterium-suspension using a disposable syringe without a needle. Infiltrated leaves were harvested 6 days post-infiltration. Plants can be screened for the presence of target antigen expression by assessment of lichenase activity assay and immunoblot analysis (FIGS. 6 and 7). Zymogram analysis revealed the expression of NA chimeric proteins in the Nicotiana benthamiana transgenic roots tested. The expression is associated with lichenase activity (FIG. 6). The activity band related to the fusion proteins show a higher molecular weight than the lichenase control and the same molecular weight of the product expressed by plants after agro-infection, confirming the presence of whole fusion product.

### Clonal Root and Clonal Root Line Generation

Nicotiana benthamiana leaf explants 1 cm×1 cm wide are obtained from leaves after sterilization in 0.1% NH₄Cl and six washing in sterile dH₂O. The explants are slightly damaged with a knife on the abacsial side and co-cultured with the Agrobacterium rhizogenes, strain A4, containing either the pBID4-Lic-HA-KDEL or the pBID4-Lic-NA-KDEL. The explants are incubated for 2 minutes with an Agrobacterium O.N. culture (O.D._{600 nm}=0.8-1) centrifuged for 10 minutes at 3000 rpm at 4° C. and resuspended in MMA medium to a final O.D._{600 nm}=0.5 in presence of 20 mM acetosyringone. At the end of the incubation, the explant is dried on sterile paper and transferred onto 0.8% agar MS plates in presence of 1% glucose and 20 mM acetosyringone. Plates are parafilmed and kept at R.T. for two days. The explants are then transferred onto MS plates in presence of 500 mg/l Cefotaxim (Cif), 100 mg/l Timentin (Tim) and 25 mg/l kanamycin. After approximately 5 weeks the generation of transgenic roots is obtained from Nicotiana benthamiana leaf explants transformed with Agrobacterium rhizogenes containing the pBID4-Lic-HA-KDEL and pBID4-Lic-NA-KDEL constructs.

After transformation, hairy roots can be cut off and placed in a line on solid, hormone free K₃ medium. Four to six days later the most actively growing roots are isolated and transferred to liquid K₃ medium. Selected roots are cultured on a rotary shaker at 24° C. in the dark and clonal lines are isolated and subcultured weekly. Roots and/or clonal lines can be screened for the presence of target antigen expression by assessment of lichenase activity assay and immunoblot analysis.

### EXAMPLE 4

### Production of Antigen

100 mg samples of N. benthamiana infiltrated leaf material were harvested at 4, 5, 6 and 7 days post-infection. The fresh tissue was analysed for protein expression right after being harvested or collected at −80° C. for the preparation of subsequent crude plants extracts or for fusion protein purification.

Fresh samples were resuspended in cold PBS 1× plus protease inhibitors (Roche) in a 1/3 w/v ratio (1 ml/0.3 g of tissue) and ground with a pestel. The homogenates were boiled for 5 minutes in SDS gel loading buffer and then clarified by centrifugation for 5 minutes at 12,000 rpm at 4° C. The supernatants were transferred in a fresh tube, and 20 μl, 1 μl or dilutions thereof were separated on a 12% SDS-PAGE and analyzed by Western analysis using anti-His₆-HA mouse or rabbit anti-lichenase polyclonal antibodies and/or by zymogram analysis to assess proteolytic activity indicating functional lichenase activity. Zymography is an electrophoretic method for measuring proteolytic activity. The method is based on a sodium dodecyl sulfate gel impregnated with a protein substrate which is degraded by the proteases resolved during the incubation period. The staining of the gel reveals sites of proteolysis as white bands on a dark blue background. Within a certain range the band intensity can be related linearly to the amount of protease loaded.

HA expression in Nicotiana benthamiana plants infiltrated either with Agrobacterium tumefaciens or Agrobacterium rhizogenes containing the plasmid pBID4-Lic-HA-KDEL leads to a specific band corresponding to the molecular weight of the chimeric protein Lic-HA-KDEL if the HA protein electrophoretic mobility in the fusion protein corresponds to the theoretic MW (the lichenase enzyme MW is about 21-24 kD).

Quantification of the chimeric protein Lic-NA-KDEL expressed in crude extract can be made by immunoblotting both on manually infiltrated tissues and on vacuum-infiltrated tissues.

### Purification of Antigens

Leaves from plants infiltrated with recombinant Agrobacterium tumefaciens containing the pBID4-Lic-HA-KDEL and pBID4-Lic-NA-KDEL constructs were ground by homogenization. Extraction buffer with “EDTA-free” protease inhibitors (Roche) and 1% Triton X-100 was used at a ratio of 3× (w/v) and rocked for 30 minutes at 4° C. Extracts were clarified by centrifugation at 9000×g for 10 minutes at 4° C. The supernatant was sequentially filtered through Mira cloth, centrifuged at 20.000×g for 30 minutes at 4° C. and filtered through 0.45-μm filter, before chromatographic purification.

Resulting extract was cut using ammonium sulfate precipitation. Briefly, (NH₄)₂SO₄ was added to extract to 20% saturation, incubated on ice for 1 hour and spun down at 18,000×g for 15 minutes. Pellet was discarded and (NH₄)₂SO₄ was added slowly to 60% saturation, incubated on ice for 1 hour, and spun down at 18,000×g for 15 minutes. Supernatant was discarded and resulting pellet was resuspended in buffer then maintained on ice for 20 minutes, followed by centrifugation at 18,000×g for 30 minutes. Supernatant was dialyzed overnight against 10,000 volumes of washing buffer:

His-tagged Lic-HA-KDEL and Lic-NA-KDEL chimeric proteins were purified by using Ni-NTA sepharose (“Chelating Sepharose Fast Flow Column,” Amersham) at room temperature under gravity. The purification was performed under non-denaturing conditions. Proteins were collected as 0.5 ml fractions, which were unified, added with 20 mM EDTA, dialyzed against 1×PBS overnight at 4° C. and analyzed by SDS-PAGE.

Alternatively, fractions were then collected together added with 20 mM EDTA, dialyzed against 10 mM NaH₂PO₄ overnight at 4° C. and purified by Anion Exchange Chromatography. For LIC-HA-KDEL and LIC-NA-KDEL purification, anion exchange column Q Sepharose Fast Flow (Amersham Pharmacia Biosciences) was used. Samples of the Lic-HA-KDEL or Lic-NA-KDEL affinity or ion-exchange purified chimeric proteins were separated on 12% polyacrylamide gels followed by Coomassie staining. Membranes were also electrophoretically transferred onto nitrocellulose membranes for Western analysis using polyclonal anti-lichenase antibody and successively with anti-rabbit IgG horseradish peroxidase-conjugated antibody.

Collected fractions after dialysis were analyzed by immunoblotting using both the pAb α-lichenase and the pAb α-anti-His₆. The His-tag was maintained by the expressed chimeric proteins and the final concentration of the purified protein was evaluated by software.

### EXAMPLE 5

### Derivation of a Murine Hybridoma Secreting Monoclonal Antibody

(Influenza A/Vietnam/03 H5N1) (NIBRG-14). NIBRG-14 is an H5N1 virus derived by reverse genetics and reassortment on the PR8 background, described in the attached document from the National Institute for Biological Standards and Controls.

A 10 week old female A/J mouse was injected intraperitoneally with plant-expressed vaccine material comprised of 50 μg of full-length N1 neuraminidase. Soluble protein was delivered in 300 μl with no adjuvant. Identical doses were given 14 days and 24 days later.

72 hours following the second boost 45 million spleen cells were fused with 5 million P3XAg8.653 murine myeloma cells using polyethylene glycol. The resulting 50 million fused cells were plated at 5×10⁵ cells per well in 10×96 well plates. HAT (hypoxanthine, aminopterin, and thymidine) selection followed 24 hours later and continued until colonies arose. All immunoglobulin-secreting hybridomas were subcloned by 3 rounds of limiting dilution in the presence of HAT.

Potential hybridomas were screened on ELISA plates for IgG specific for either LicKM (500 ng/well) or Influenza A/Vietnam/03 (300 ng of propriolactone-inactivated virus/well). Hybridoma 2B9 had a very high specific signal. The specificity of this monoclonal antibody was tested further by ELISA against plant-expressed antigens. Supernatant from 10⁶ cells, cultured for 48 hours in 2.5 ml of Iscoves minimally essential medium supplemented with 10% fetal bovine serum, was strongly reactive against NIBRG-14, and N1 neuraminidase, but not against N2 neuraminidase. The isotype of this monoclonal has yet to be defined. Frozen stocks are kept in a liquid nitrogen tank hereafter labeled as “Fraunhofer 2B9.” Sequences of the 2B9 anti-N1 monoclonal antibody light and heavy chain variable regions are presented in Appendix A.

### EXAMPLE 6

### Characterization of Inhibition Activity of Antibody

For characterization of antibody activity, we used an assay based on the recommended WHO neuraminidase assay protocol, with minor modifications. For each assay, reactions were conducted in triplicate and consisted of:

a. 1 μl of fresh extract prepared from plant tissue that had been infiltrated with an expression vector encoding neuraminidase (N1) lacking the N-terminal transmembrane domain. For the purposes of preparing the plant extract, 1 μl of buffer was used for each mg of plant tissue.

b. No antibody (positive control) or a volume of monoclonal antibody (either Ab αN1 [from hybridoma 2B9] or Ab RSV [antibody against viral RSV F protein raised in mouse]) such that the molar ratio of neuraminidase to antibody was 1:1, 1:10, 1:20 or 1:30.

Note that the neuraminidase antibody and RSV F antibody are of the same isotype (murine IgG 2b). Reactions were incubated at room temperature for 30 minutes to allow the antibodies opportunity to recognize the plant-produced neuraminidase. Reactions were then incubated at 37° C., an optimum temperature for neuraminidase activity. Product (sialic acid) accumulation was assessed colormetrically at 549 nm using a spectrophotometer, and quantified against sialic acid standards.

Percentage of neuraminidase inhibition was calculated using the equation:

% inhibition=([PC−Tr]/PC)×100

where: PC—neuraminidase activity of the positive control

Tr—neuraminidase activity of antibody/neuraminidase combination.

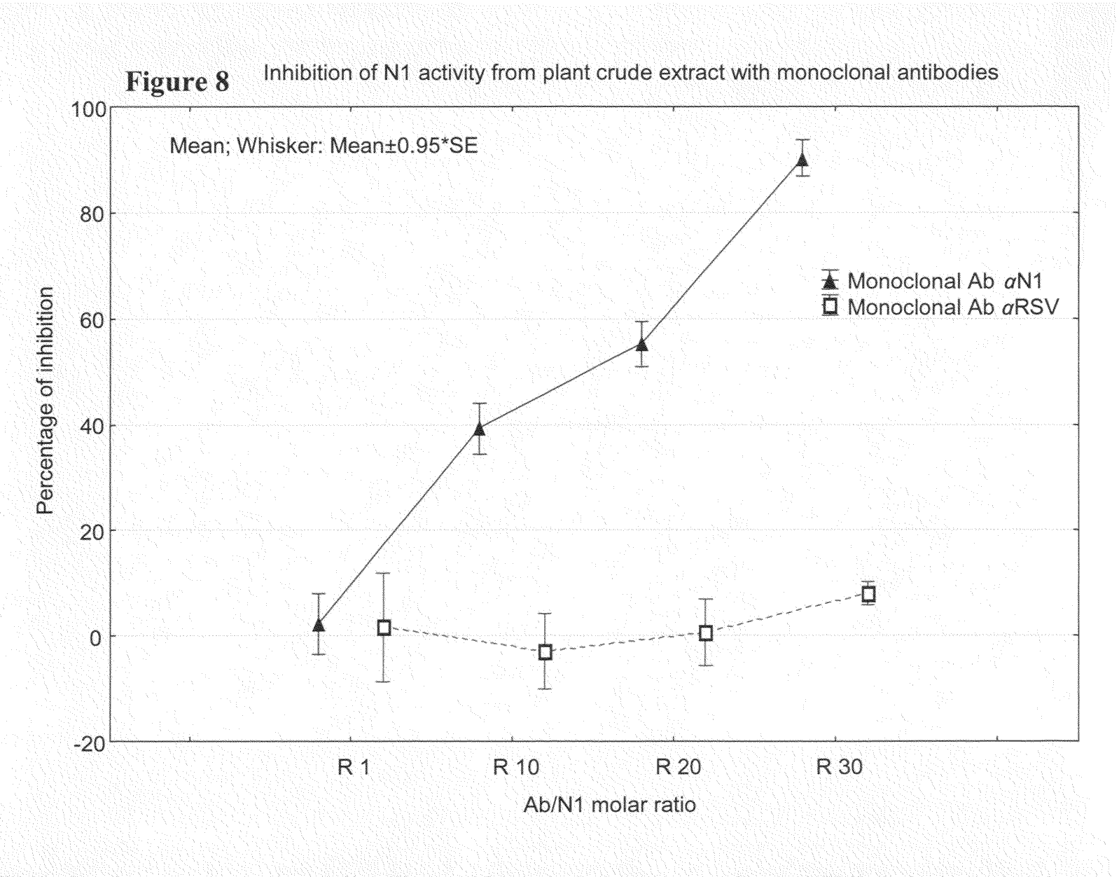

A molar comparison of the antibody's ability to inhibit the neuraminidase is depicted in FIG. 8. Percent neuraminidase inhibition (calculated according equation above) is shown on the y-axis and the molar ratio of neuraminidase to antibody (1:1, 1:10, 1:20 or 1:30) is shown on the x-axis as R1, R10, R20 or R30, respectively (FIG. 8). Standard errors are shown for p<0.05.

Inhibition of plant-expressed neuraminidase activity was seen in the presence of the murine monoclonal antibody that was generated against this same plant expressed neuraminidase. By comparison, the inability of an unrelated (RSV) antibody to inhibit the same plant produced neuraminidase is also shown (FIG. 8).

In order to determine whether anti-NA 2B9 is capable of recognizing N1 antigens from influenza strains besides the strain from which the 2B9 antigen was originally derived, we performed neuraminidase assays on five other strains: three different H5N1 strains: A/Vietnam/1203/04, A/Hong Kong/156/97, and A/Indonesia/05; and one H1N1 strain: A/New Caldonia/99. We also performed the HI assays on one H3N2 strain (A/Udorn/72) to determine whether anti-NA 2B9 were capable of recognizing a subtype other than N1.

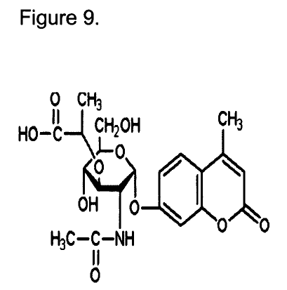

In these experiments, NA inhibition was measured using 2′-(4-Methylumbelliferyl)-α-D-N-acetylneuraminic acid (MDNA, FIG. 9), which liberates a quantifiable fluorescent tag in response to sialidase activity. MDNA has absorption and fluorescence emission maxima of approximately 365 and 450, respectively, and signal can be detected fluorometrically with a sensitivity as low as 10⁶ virus particles/ml (10⁴ particles total) with a broad linear range of 0-30 fold dilutions of the virus stock. The system used amplified live virus which was diluted to the appropriate concentration in reaction buffer (100 mM sodium acetate, pH 6.5, 10 mM CaCl₂) and added directly to plates containing 2-fold serial dilutions of the tested antibody. Because active NA is located on the viral surface, no purification of NA protein was necessary to measure enzymatic activity. The antibody was 2-fold serially-diluted and aliquoted in triplicate into 384 microplate wells (Table 1). Titrated virus (also diluted in reaction buffer) was added to the plate wells, followed by a 30 minute incubation. Afterward, MDNA was diluted to 0.2 mM in reaction buffer and added to the plate wells, and the reaction was allowed to proceed for and additional 30 minutes. The reaction was stopped with the addition of 200 mM sodium carbonate, pH 9.5.

TABLE 1
Plate Layout for Triplicate NA Assays

| | | μg/ml antibody | | | | | | | | | | |
|---|---|---|---|---|---|---|---|---|---|---|---|---|
| | | 250 | 125 | 67 | 33 | 16 | 8 | 4 | 2 | 1 | 0 | Tamiflu |
| A/Udorn/72 | replicate 1 | A | A | A | A | A | A | A | A | A | A | 2 μM |
| | replicate 2 | B | B | B | B | B | B | B | B | B | B | 2 μM |
| | replicate 3 | C | C | C | C | C | C | C | C | C | C | 2 μM |
| A/New Caldonia/99 | rep 1 | A | A | A | A | A | A | A | A | A | A | 2 μM |
| | replicate 2 | B | B | B | B | B | B | B | B | B | B | 2 μM |
| | replicate 3 | C | C | C | C | C | C | C | C | C | C | 2 μM |
| A/Vietnam/04 | replicate 1 | A | A | A | A | A | A | A | A | A | A | 2 μM |
| | replicate 2 | B | B | B | B | B | B | B | B | B | B | 2 μM |
| | replicate 3 | C | C | C | C | C | C | C | C | C | C | 2 μM |
| A/Hong Kong/97 | rep 1 | A | A | A | A | A | A | A | A | A | A | 2 μM |
| | replicate 2 | B | B | B | B | B | B | B | B | B | B | 2 μM |
| | replicate 3 | C | C | C | C | C | C | C | C | C | C | 2 μM |
| A/Indonesia/05 | rep1 | A | A | A | A | A | A | A | A | A | A | 2 μM |
| | replicate 2 | B | B | B | B | B | B | B | B | B | B | 2 μM |
| | replicate 3 | C | C | C | C | C | C | C | C | C | C | 2 μM |

| | | | |
|---|---|---|---|
| Control - No virus | 1 | 1 | 1 |

Titration of each cell-culture amplified virus strain was performed prior to the assay to establish the linear range of NA activity detection and the minimal virus concentration necessary for a signal window of 10-15. Oseltamivir carboxylate (Tamiflu®, 2 μM) was used as the control drug for this assay. Oseltamivir carboxylate is a specific inhibitor of influenza virus NA activity and is privately available from the SRI chemical respository.

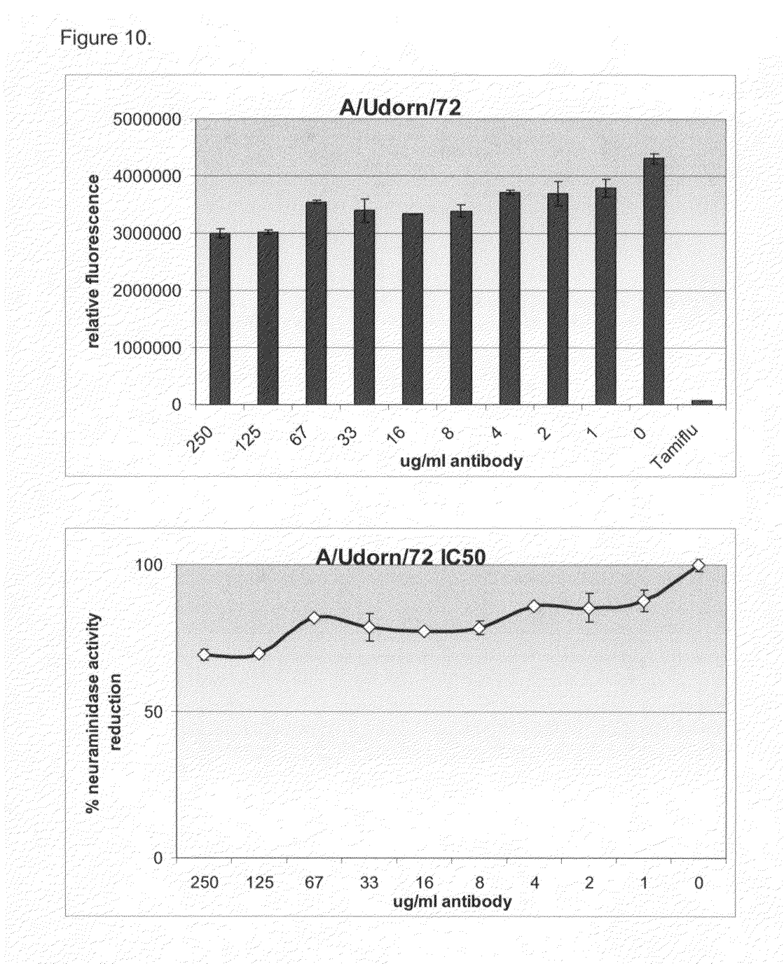

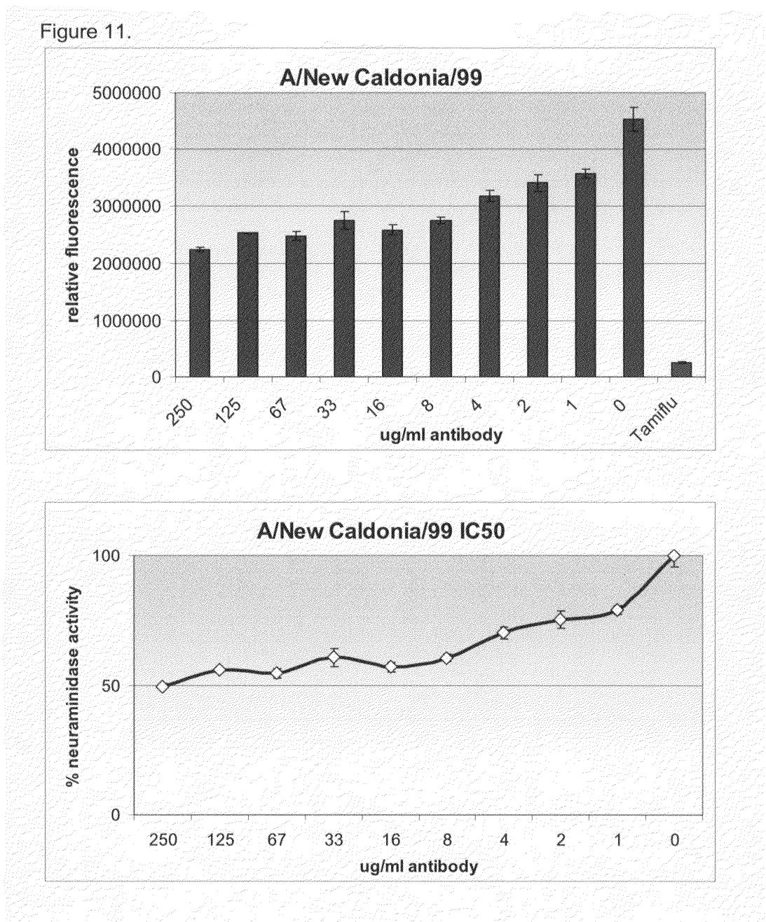

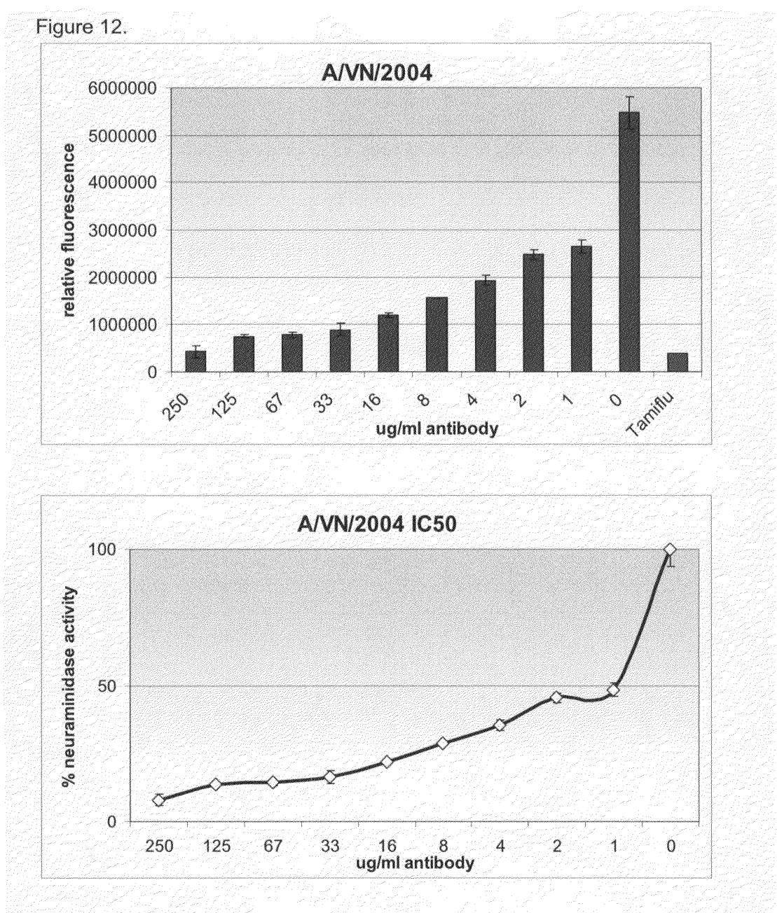

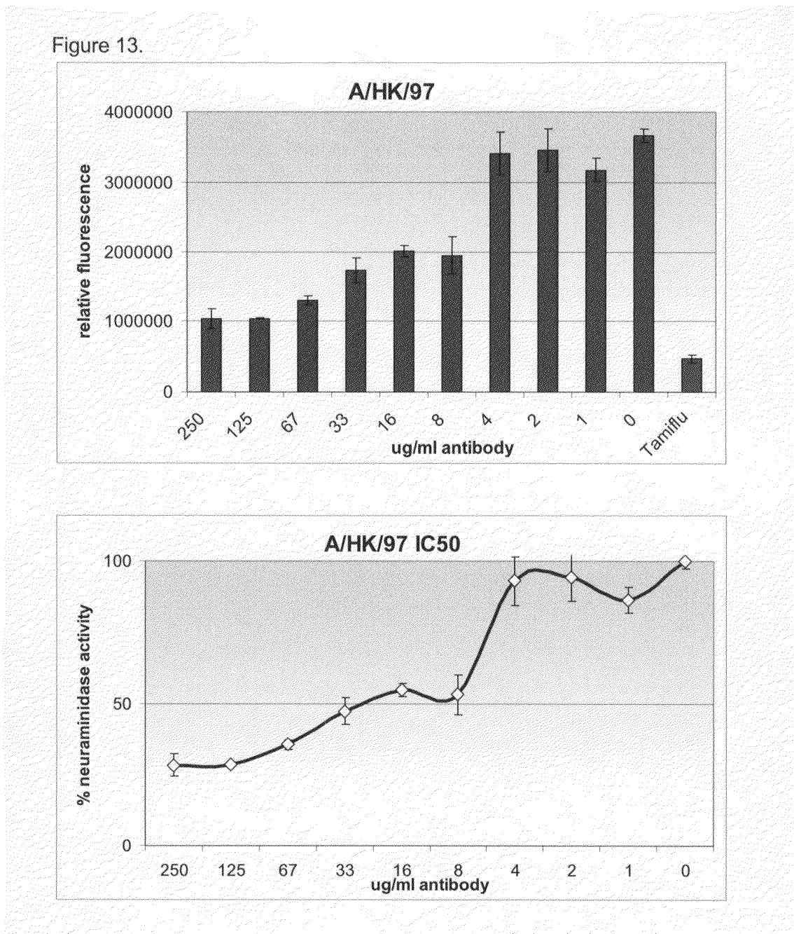

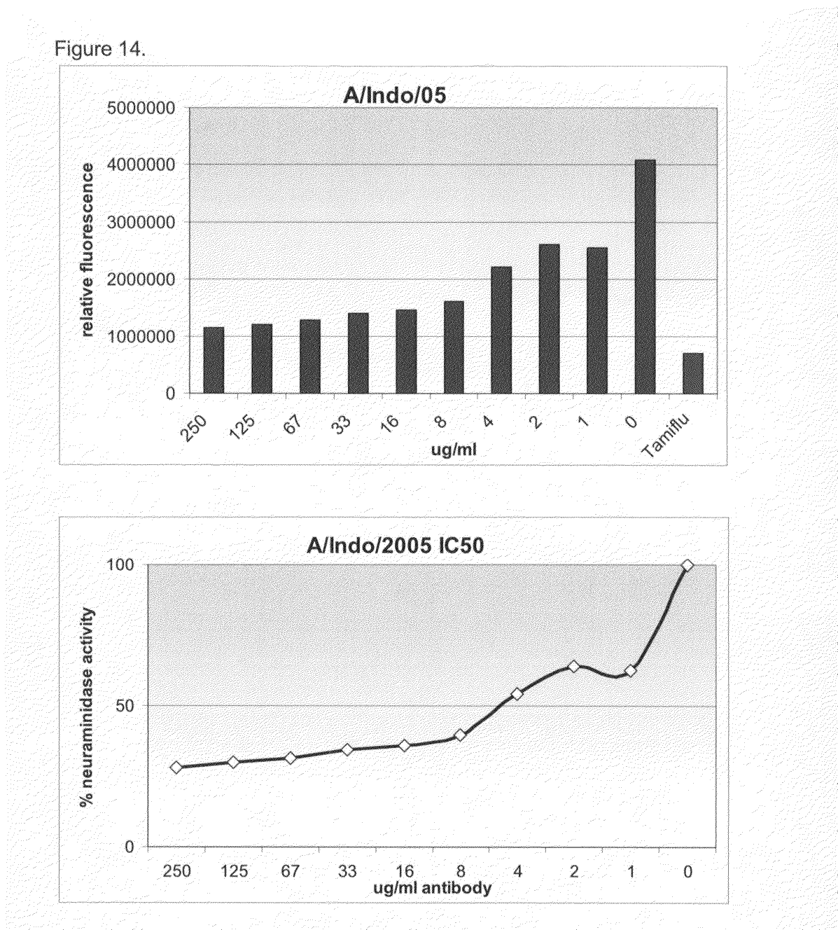

Antibody dilutions and controls were run in triplicate assays (Table 2). Antibody concentrations from 1-250 μg/ml (final well volume) were tested, and oseltamivir carboxylate (2 μM final well concentration) was included as a positive inhibition control. A summary of the 50% results for each virus strain are presented in Table 2. Column and linear graphs comparing efficacy with oseltamivir carboxylate and demonstrating IC₅₀ are shown in FIGS. 10-14. Calculated numbers are shown in Appendix B. Raw data are shown in Appendix C.

TABLE 2
NA assay IC₅₀* results 01-12-2007

| Virus | Antibody IC₅₀ μg/ml |
|---|---|
| A/Udorn/72 | N/D* |
| A/NC/99 | 125-250 |
| A/VN/04 | <1 |
| A/HK/97 | 16-33 |
| A/Indo/05 | 4-8 |

*IC₅₀ = 50% inhibitory concentration
**N/D = not determined

1. An isolated monoclonal antibody or antigen binding fragment thereof which binds neuraminidase, wherein the antibody is capable of inhibition of neuraminidase enzyme activity.

2. The antibody of claim 1, wherein the antibody is an IgG antibody.

3. The antibody of claim 1, wherein the antibody is an IgG2b antibody.

4. The antibody of claim 1, wherein the antibody is an antigen-binding fragment of an antibody.

5. The antibody of claim 4, wherein the antibody is an scFv, Fv, Fab′, Fab, diabody, linear antibody or F(ab′).sub.2 antigen-binding fragment of an antibody.

6. The antibody of claim 4, wherein the antibody is a CDR, univalent fragment, single domain antibody.

7. The antibody of claim 1, wherein the antibody is a human, humanized or part-human antibody or antigen-binding fragment thereof.

8. The antibody of claim 17, wherein the antibody comprises an antigen-binding region of the antibody operatively attached to a human antibody framework or constant region.

9. The antibody of claim 1, wherein the antibody is a chimeric antibody.

10. The antibody of claim 1, wherein the antibody is a bispecific antibody.

11. The antibody of claim 1, wherein the antibody is a recombinant antibody.

12. The antibody of claim 1, wherein the antibody is an engineered antibody.

13. The antibody of claim 1, wherein the antibody is prepared by a process comprising immunizing an animal with purified neuraminidase and selecting from the immunized animal an antibody that binds to neuraminidase and effectively competes with the monoclonal antibody produced by hybridoma 2B9 for binding to neuraminidase.

14. The antibody of claim 1, wherein the antibody has the ability to inhibit neuraminidase enzyme activity.

15. The antibody of claim 1, wherein the antibody is the monoclonal antibody produced by the hybridoma 2B9.

16. The antibody of any one of claims 1 through 15, wherein the antibody is operatively attached to at least a first biological agent or diagnostic agent.

17. The antibody of claim 16, wherein the antibody is operatively attached to at least a first agent that cleaves a substantially inactive prodrug to release a substantially active drug.

18. The antibody of claim 17, wherein the antibody is operatively attached to alkaline phosphatase that cleaves a substantially inactive phosphate-prodrug to release a substantially active anti-viral drug.

19. The antibody of claim 18, wherein the anti-viral drug is an anti-influenza agent.

20. The antibody of claim 16, wherein the antibody is operatively attached to at least a first anti-viral agent.

21. The antibody of claim 20, wherein the antibody is operatively attached to an anti-influenza agent.

22. The antibody of claim 16, wherein the antibody is operatively attached to a diagnostic, imaging or detectable agent.

23. The antibody of claim 22, wherein the antibody is operatively attached to an X-ray detectable compound, a radioactive ion or a nuclear magnetic spin-resonance isotope.

24. The antibody of claim 23, wherein the antibody is operatively attached to: (a) the X-ray detectable compound bismuth (III), gold (III), lanthanum (III) or lead (II); (b) the detectable radioactive ion copper⁶⁷, gallium^{67,} gallium⁶⁸, indium¹¹¹, indium¹¹³, iodine¹²³, iodine¹²⁵, iodine¹³¹, mercury¹⁹⁷, mercury²⁰³, rhenium¹⁸⁶, rhenium¹⁸⁸, rubidium⁹⁷, rubidium¹⁰³, technetium^{99m} or yttrium⁹⁰; or (c) the detectable nuclear magnetic spin-resonance isotope cobalt (II), copper (II), chromium (III), dysprosium (III), erbium (III), gadolinium (III), holmium (III), iron (II), iron (III), manganese (II), neodymium (III), nickel (II), samarium (III), terbium (III), vanadium (II) or ytterbium (III).

25. The antibody of claim 22, wherein the antibody is operatively attached to biotin, avidin or to an enzyme that generates a colored product upon contact with a chromogenic substrate.

26. The antibody of claim 16, wherein the antibody is operatively attached to the biological agent as a fusion protein prepared by expressing a recombinant vector that comprises, in the same reading frame, a DNA segment encoding the antibody operatively linked to a DNA segment encoding the biological agent.

27. The antibody of claim 16, wherein the antibody is operatively attached to the biological agent via a biologically releasable bond or selectively cleavable linker.

28. The antibody of claim 1, wherein the composition is a pharmaceutically acceptable composition that further comprises a pharmaceutically acceptable carrier.

29. The composition of claim 28, wherein the pharmaceutically acceptable composition is formulated for parenteral administration.

30. The composition of claim 28, wherein the pharmaceutically acceptable composition is a formulation of a plant produced antibody.

31. The composition of claim 28, wherein the pharmaceutically acceptable composition is an encapsulated or liposomal formulation.

32. The composition of claim 28, wherein the composition further comprises a second therapeutic agent.

33. A method for treating a influenza infection, comprising administering to an animal in need thereof a biologically effective amount of the composition of claim 1, thereby treating influenza infection.

34. Use of an antibody according to claim 1, for the diagnosis of a condition due to infection by a human influenza virus, or for typing a human influenza virus.

35. An assay for determining the presence of human influenza virus in a sample using substances according to claim 1.

36. A hybridoma that produces an antibody or an antigen-binding fragment according to claim 1.

37. The hybridoma of claim 36 which is hybridoma 2B9.

38. The hybridoma of claim 36, wherein the antibody or antigen-binding fragment has the same antigenic specificity of an antibody produced by hybridoma 2B9.


SEQUENCE LISTING (12 sequences)

SEQ ID NO: 1 - protein, 449 aa, Artificial Sequence
  Influenza Proteins
MNPNQKIITIGSICMVTGIVSLMLQIGNMISIWVSHSIHTGNQHQSEPISNTNLLTEKAVASVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPDAGEITCVCRDNWHGSNRPWVSFNQNLEYQIGYICSGVFGDNPRPNDGTGSCGPVSSNGAGGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 2 - protein, 469 aa, Artificial sequence
  Influenza proteins
MNPNQKIITIGSVSLTISTICFFMQIAILITTVTLHFKQYEFNSPPNNQVMLCEPTIIERNITEIVYLTNTTIEKEICPKLAEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDENATASFIYNGRLVDSIVSWSKKILRTQESECVCINGTCTVVMTDGSASGKADTKILFIEEGKIVHTSTLSGSAQHVEECSCYPRYPGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKQETEVLWTSNSIVVFCGTSGTYGTGSWPDGADINLMPI

SEQ ID NO: 3 - protein, 21 aa, Artificial sequence
  Influenza proteins
MNPNQKIITIGSICMVTGIVS

SEQ ID NO: 4 - protein, 428 aa, Artificial sequence
  Influenza peptides
LMLQIGNMISIWVSHSIHTGNQHQSEPISNTNLLTEKAVASVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPDAGEITCVCRDNWHGSNRPWVSFNQNLEYQIGYICSGVFGDNPRPNDGTGSCGPVSSNGAGGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 5 - protein, 37 aa, Artificial sequence
  Influenza peptides
MNPNQKIITIGSVSLTISTICFFMQIAILITTVTLHF

SEQ ID NO: 6 - protein, 432 aa, Artificial sequence
  Influenza peptides
KQYEFNSPPNNQVMLCEPTIIERNITEIVYLTNTTIEKEICPKLAEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDENATASFIYNGRLVDSIVSWSKKILRTQESECVCINGTCTVVMTDGSASGKADTKILFIEEGKIVHTSTLSGSAQHVEECSCYPRYPGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKQETEVLWTSNSIVVFCGTSGTYGTGSWPDGADINLMPI

SEQ ID NO: 7 - DNA, 1446 bp, Artificial sequence
  Infuenza virus neuraminidase
ggatccttaattaaaatgggattcgtgcttttctctcagcttccttctttccttcttgtgtctactcttcttcttttccttgtgatttctcactcttgccgtgctcaaaatgtcgaccttatgcttcagattggaaacatgatttctatttgggtgtcacactctattcacactggaaaccagcatcagtctgagccaatttctaacactaaccttttgactgagaaggctgtggcttctgttaagttggctggaaactcttctctttgccctattaacggatgggctgtgtactctaaggataactctattaggattggatctaagggagatgtgttcgtgattagggagccattcatttcttgctctcaccttgagtgccgtactttcttccttactcagggtgctcttcttaacgataagcactctaacggaactgtgaaggataggtctccacacaggactcttatgtcttgtccagttggagaagctccatctccatacaactctagattcgagtctgttgcttggagtgcttctgcttgccatgatggaacttcatggcttactattggaatttctggaccagataacggagctgttgctgtgcttaagtacaacggaattattactgataccatcaagtcttggaggaacaacattcttaggactcaggagtctgagtgtgcttgcgttaacggatcttgcttcactgtgatgactgatggaccatctaatggacaggcttctcacaagattttcaagatggagaagggaaaggttgtgaagtctgtggaacttgatgctccaaactaccattacgaggagtgttcttgctatccagatgctggagagattacttgtgtgtgccgtgataattggcatggatctaacaggccatgggtgtcattcaatcagaaccttgagtaccagattggttacatttgctctggagtgttcggagataatccaaggccaaacgatggaactggatcttgtggaccagtgtcatctaatggagctggaggagtgaagggattctctttcaagtacggaaacggagtttggattggaaggactaagtctactaactctaggagtggattcgagatgatttgggacccaaacggatggactgagactgattcttctttctctgtgaagcaggatattgtggctattactgattggagtggatactctggatctttcgttcagcacccagagcttactggacttgattgcattaggccatgcttctgggttgaacttattaggggaaggccaaaggagtctactatttggacttctggatcttctatttctttctgcggagtgaattctgatactgtgggatggtcttggccagatggagctgagcttccattcactattgataaggtcgaccatcatcatcatcaccacaaggatgagctttgactcgag

SEQ ID NO: 8 - DNA, 1458 bp, Artificial sequence
  Influenza virus neuraminidase
ggatccttaattaaaatgggattcgtgcttttctctcagcttccttctttccttcttgtgtctactcttcttcttttccttgtgatttctcactcttgccgtgctcaaaatgtcgacaagcagtacgagttcaactctccaccaaacaaccaggttatgctttgcgagccaactattattgagaggaacattactgagattgtgtaccttactaacactactattgagaaggagatttgcccaaagttggctgagtaccgtaattggtctaagccacagtgcaacattactggattcgctccattctctaaggataactcaattaggctttctgctggaggagatatttgggttacaagggagccatacgtttcttgcgatccagataagtgctaccagttcgctcttggacaaggaactactcttaacaacgtgcactctaacgatactgtgcacgataggactccataccgtactcttttgatgaacgagcttggagttccattccaccttggaactaagcaagtgtgcattgcttggtcatcttcatcttgccacgatggaaaggcttggcttcatgtttgcgtgactggagatgatgagaacgctactgcttctttcatctacaacggaaggcttgtggattctattgtttcttggtctaagaagattcttaggactcaggagtctgagtgtgtgtgcattaacggaacttgcactgtggttatgactgatggatctgcttctggaaaggctgatacaaagattcttttcattgaggagggaaagattgtgcacacttctactctttctggatctgctcagcatgttgaggagtgttcttgctacccaaggtatccaggagttagatgtgtgtgccgtgataactggaagggatctaacaggccaattgtggatattaacattaaggattactctattgtgtcatcttatgtgtgctctggacttgttggagatactccaaggaagaacgattcttcttcatcttcacactgccttgatccaaataacgaggagggaggacatggagttaagggatgggctttcgatgatggaaacgatgtttggatgggaaggactatttctgagaagttgaggagcggatacgagactttcaaagtgattgagggatggtctaacccaaattctaagctgcagattaacaggcaagtgattgtggataggggaaacaggagtggatactctggaattttctctgtggagggaaagtcttgcattaacagatgcttctacgtggagcttattaggggaaggaagcaggagactgaggttttgtggacttctaactctattgtggtgttctgcggaacttctggaacttacggaactggatcttggccagatggagctgatattaaccttatgccaattgtcgaccatcatcaccatcaccacaaggatgagctttgactcgag

SEQ ID NO: 9 - DNA, 452 bp, Artificial sequence
  Monoclonal antibody light and heavy chain
atggaatggagctgggtctttctctttctcctgtcagtaactgcaggtgtccactcccaggtccagctgcagcagtctggagctgagctggtaaggcctgggacttcagtgaagatgtcctgcaaggctgctggatacaccttcactaactactggataggttgggtaaaacagaggcctggacatggccttgagtggattggagatatttaccctgaaaatgatttttctaactacaatgagaagttcaaggacaaggccacactgactgcagacacatcctccagaacagcctacatgcagctcagcagcctgacatctgaggactctgccatctattactgtgtaagagcgaatgagggctggtacttagatgtctggggcacagggaccacggtcagtgtctcctcagccaaaacaacacccccacccgtctatccattggcccctgg

SEQ ID NO: 10 - protein, 150 aa, Artificial sequence
  Monoclonal antibody light and heavy chain
MEWSWVFLFLLSVTAGVHSQVQLQQSGAELVRPGTSVKMSCKAAGYTFTNYWIGWVKQRPGHGLEWIGDIYPENDFSNYNEKFKDKATLTADTSSRTAYMQLSSLTSEDSAIYYCVRANEGWYLDVWGTGTTVSVSSAKTTPPPVYPLAP

SEQ ID NO: 11 - DNA, 426 bp, Artificial sequence
  Monoclonal antibody light and heavy chain
atgaggtacccggctcagcttctgaggttgctgctggtgtggcttacaggtgccagatgtgacatccagatgactcagtctccagcctccctatctgaatctgtgggagaaactgtcaccatcacatgtcgagcaagtgagaatatttacagttatttagcatggtatcagcagaaacagggaaaatctcctcagctcctggtctattttgcaaaaaccttagcagaaggtgtgccatcaacgttcagtggcagtggatcaggcacactgttttctctgaagatcaacagcctgcagcctgaagattttgggaattattactgtcaacatcattatggcactccgtacacgttcggaggggggaccaagctggaaataaaacgggctgatgctgcaccaactgtatccatcttcccaccatccagt

SEQ ID NO: 12 - protein, 142 aa, Artificial
  Monoclonal antibody light and heavy chain
MRYPAQLLRLLLVWLTGARCDIQMTQSPASLSESVGETVTITCRASENIYSYLAWYQQKQGKSPQLLVYFAKTLAEGVPSTFSGSGSGTLFSLKINSLQPEDFGNYYCQHHYGTPYTFGGGTKLEIKRADAAPTVSIFPPSS